SEQUENCE LISTING (11 sequences)

SEQ ID NO: 1 - protein, 1255 aa, Homo sapiens [sequence omitted]

SEQ ID NO: 2 - protein, 10 aa, artificial
  anti-HER2 antibody HCDR1
GFNIKDTYIH

SEQ ID NO: 3 - protein, 17 aa, Artificial
  anti-HER2 antibody HCDR2
RIYPTNGYTRYADSVKG

SEQ ID NO: 4 - protein, 11 aa, Artificial
  anti-HER2 antibody HCDR3
WGGDGFYAMDY

SEQ ID NO: 5 - protein, 11 aa, Artificial
  anti-HER2 antibody LCDR1
RASQDVNTAVA

SEQ ID NO: 6 - protein, 7 aa, Artificial
  anti-HER2 antibody LCDR2
SASFLYS

SEQ ID NO: 7 - protein, 9 aa, Artificial
  anti-HER2 antibody LCDR3
QQHYTTPPT

SEQ ID NO: 8 - protein, 120 aa, Artificial [sequence omitted]

SEQ ID NO: 9 - protein, 107 aa, Artificial [sequence omitted]

SEQ ID NO: 10 - protein, 450 aa, Artificial
  anti-HER2 antibody Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 214 aa, Artificial
  anti-HER2 antibody Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2020/0061190 A1
(43) Pub. Date: Feb. 27, 2020

(54) ANTIBODY FORMULATION

(71) Applicant: GLENMARK PHARMACEUTICALS S.A., La Chaux-de-Fonds (CH)

(72) Inventors: Jonathan André ALBANESE, La Chaux-de-Fonds (CH); Roberto Pier-Lorenzo GIOVANNINI, La Chaux-de-Fonds (CH); Kevin Niall O'MAHONY, La Chaux-de-Fonds (CH)

(21) Appl. No.: 16/504,411

(22) Filed: Jul. 8, 2019

Related U.S. Application Data
(63) Continuation of application No. 16/199,501, filed on Nov. 26, 2018, now abandoned.
Application No. 16/199,501 is a continuation of application No. 14/654,096, filed on Jun. 19, 2015, now abandoned.
(60) Provisional application No. 61/745,293, filed on Dec. 21, 2012.

(51) Int. Cl. A61K 39/395; A61K 47/12; A61K 47/26; A61K 47/18; A61K 47/02; A61K 47/22; C07K 16/32

(52) U.S. Cl. CPC A61K 39/39591; A61K 47/12; A61K 47/26; A61K 47/183; C07K 2317/76; A61K 47/22; C07K 16/32; C07K 2317/24; C07K 2317/94; A61K 47/02

(57) ABSTRACT

A pharmaceutical formulation is described comprising a therapeutically effective amount of an antibody, an acetate buffer, a lyoprotectant, a bulking agent and a surfactant.

### FIELD OF THE INVENTION

The invention relates to a pharmaceutical formulation comprising an anti-HER2 antibody. Preferably, the pharmaceutical formulation comprises a lyophilized anti-HER2 antibody formulation. More preferably the formulation comprises a stable lyophilized anti-HER2 antibody formulation that has been reconstituted with a diluent to generate a stable reconstituted formulation.

### BACKGROUND OF THE INVENTION

The ErbB receptor family is composed of four plasma membrane-bound receptor tyrosine kinases: EGFR/ErbB-1, HER2/ErbB-2, HER3/ErbB-3, and HER4/ErbB-4. Both homo- and heterodimers are formed by the four members of the EGFR family, with HER2 being the preferred and most potent dimerization partner for other ErbB receptors (Graus-Porta D et al., (1997) Embo J, 16: 1647-1655; Tao R H et al., (2008) J Cell Sci, 121: 3207-3217). All four receptors contain an extracellular ligand binding domain, a transmembrane domain, and an intracellular domain that can interact with a multitude of signalling molecules and exhibit both ligand-dependent and ligand-independent activity. HER2 (Human Epidermal Growth Factor Receptor 2) also known as Neu, ErbB-2, CD340 (cluster of differentiation 340) or p185 is a protein that in humans is encoded by the ERBB2 gene. HER2 activation leads to receptor phosphorylation, which triggers a cascade of downstream signals through multiple signalling pathways, such as MAPK, phosphoinositol 3-kinase/AKT, JAK/STAT and PKC, which ultimately results in the regulation of multiple cellular functions, such as growth, survival and differentiation (Huang Z et al., (2009) Expert Opin Biol Ther, 9: 97-110). Over-expression of this gene has been shown to play an important role in the pathogenesis and progression of certain aggressive types of breast cancer.

Amplification or over-expression of the ERBB2 gene occurs in approximately 30% of breast cancers. It is strongly associated with increased disease recurrence and a worse prognosis (Roy V & Perez E A (2009) Oncologist, 14(11): 1061-9). Over-expression is also associated with other human cancer types including: prostate cancer, non-small cell lung cancer, bladder cancer, ovarian cancer, gastric cancer, colon cancer, oesophageal cancer, squamous cell carcinoma of the head and neck and aggressive forms of uterine cancer, such as uterine serous endometrial carcinoma (Garcia de Palazzo I et al., (1993) Int J Biol Markers, 8: 233-239; Ross J S et al., (2003) Oncologist, 8: 307-325; Osman I et al., (2005) J Urol, 174: 2174-2177; Kapitanović S et al., (1997) Gastroenterology, 112: 1103-1113; Turken O et al., (2003) Neoplasma, 50: 257-261; Oshima C T et al., (2001) Int J Biol Markers, 16: 250-254; Santin A D et al (2008) Int J Gynaecol Obstet, 102(2): 128-31).

Known drugs that target HER2 include the monoclonal antibody trastuzumab (Herceptin® which is effective in cancers over expressing HER2. Another monoclonal antibody pertuzumab (Perjeta®/Omnitarg™) inhibits dimerization of HER2 and HER3 receptors.

### SUMMARY OF THE INVENTION

According to the invention there is provided a pharmaceutical formulation comprising a formulation of an anti-HER2 antibody or a fragment thereof and one or more pharmaceutically acceptable excipients and an acetate buffer.

The applicants have discovered that use of an acetate buffer in the preparation of a liquid formulation imparts significant stability on the anti-HER2 antibody formulation and this buffer was used in the development of a lyophilized formulation and subsequently a reconstituted formulation.

Therefore the invention also relates to a lyophilized formulation comprising an anti-HER2 antibody or a fragment thereof, a lyoprotectant, a bulking agent, a surfactant and an acetate buffer, wherein the pH of the formulation is pH 5.0-6.0, and its reconstituted formulation.

### BRIEF DESCRIPTION OF THE FIGURES

FIGS. 1a-1d show the results of an accelerated stability study of an anti-HER2 antibody at 37° C. The anti-HER2 antibody was formulated with various excipients that were verified for their potential in stabilising the antibody in a liquid form. The main peak percentage results by HP-CEX are shown in FIG. 1a, the percentage of monomers by HP-SEC in FIG. 1b, the percentage of aggregation in FIG. 1c and the percentage loss of anti-HER2 antibody concentration in FIG. 1d.

FIGS. 2a-2e show the results of an accelerated stability study of an anti-HER2 antibody at 37° C., in a liquid formulation. Different concentrations of the anti-HER2 antibody were formulated at pH values in the range of pH 4.0 to pH 6.0 and with different buffers, either citrate or acetate. The main peak by HP-CEX results are shown in FIG. 2a, the percentage of monomers by HP-SEC in FIG. 2b, the percentage of aggregation in FIG. 2c, the percentage of fragmentation in FIG. 2d and the percentage loss of anti-HER2 antibody concentration in FIG. 2e.

FIGS. 3a-3c show the results of a screen to evaluate the optimal ranges of salt concentration, buffer type and antioxidant for the anti-HER2 antibody liquid formulation kept for one month at 37° C. FIG. 3a shows the aggregation and fragmentation at various NaCl concentrations at pH 5.5. The y-axis shows percentage aggregation or fragmentation. Aggregation is shown in the light grey bars, left hand side and fragmentation is shown in the dark grey bars, right hand side; for each concentration of NaCl. FIG. 3b shows anti-HER2 antibody deamidation at various NaCl concentrations at pH 5.5. FIG. 3c shows the percentage of aggregation and fragmentation of the anti-HER2 antibody when formulated with citrate or acetate at various concentrations at pH 5.5. Aggregation is shown in the light grey bars, left hand side and fragmentation is shown in the dark grey bars, right hand side; for each concentration of citrate or acetate.

FIG. 4 shows the percentage of monomers present in the 22 formulations tested (see Table 8 for details) at time 0 (light coloured bars; left-hand side) and after five cycles of freeze-thaw (dark coloured bars; right-hand side). Each formulation tested is shown on the x-axis with the percentage of monomer shown on the y-axis. Formulations 6, 8 10 and 19 were selected for further experimental work.

FIG. 5 shows the percentage of monomers present in the four formulations tested after reconstitution of the cakes. All amounts are shown as % monomers for formulations 6, 8, 10 and 19 selected from the previous screen. The column on the far right is a control at time 0.

FIG. 6 shows the percentage of monomers present in the 12 formulations tested (see Table 11 for details) at time 0 (light coloured bars; left-hand side) and after five cycles of freeze-thaw (dark coloured bars; right-hand side). Each formulation tested is shown on the x-axis with the percentage of monomer shown on the y-axis. Formulations 1, 4, 7, 10 and 11 were selected for lyophilization.

FIG. 7 shows the percentage of monomers present in 18 formulations tested (see Table 12 for details) at time 0 (light coloured bars; left-hand side) and after lyophilization and high pressure liquid chromatography-size exclusion (dark coloured bars; right-hand side). Each formulation tested is shown on the x-axis with the percentage of monomer shown on the y-axis.

FIGS. 8a-8c show the stability results for seven lyophilized formulations tested (see Table 13 for details) in a one month stability study at 40° C. FIG. 8a shows the percentage of monomers present in the formulations tested at time 0 (white bars; left-hand side), after lyophilization (light coloured bars; centre) and after one month at 40° C. (dark coloured bars; right-hand side). Each formulation tested is shown on the x-axis with the percentage of monomer shown on the y-axis. FIG. 8b shows the % main peak after cation exchange chromatography for the formulations tested at time 0 (white bars; left-hand side), after lyophilization (light coloured bars; centre) and after one month at 40° C. (dark coloured bars; right-hand side). The line connecting the columns indicates the percentage loss of main peak for each formulation after one month storage at 40° C. FIG. 8c shows the percentage of moisture present for each formulation, tested after lyophilization (light coloured bars; left-hand side) and after storage for one month at 40° C. (dark coloured bars; right-hand side).

FIGS. 9a-9b show the stability results for Formulations 1 and 2 (see Table 16 for details) in an accelerated stability study for the time periods time 0 (liquid and lyophilized formulations; clear and light grey bars respectively), one month at 40° C. (dark gray bars) and two months at 40° C. (black bars). FIG. 9a shows the percentage monomers (y-axis) present in Formulations 1 and 2 as measured by HPLC-SEC and FIG. 9b shows the HPLC-CEX percentage main peak (y-axis) for Formulations 1 and 2.

FIGS. 10a-10b show the stability results for the lyophilized anti-HER2 antibody formulation in a long term stability study for the time periods indicated on the x-axis. FIG. 10a shows the percentage monomer (y-axis) present in the formulation as measured by HPLC-SEC at the time periods of 1, 2, 3, 6, 9 and 12 months. FIG. 10b shows the HPLC-CEX percentage main pic (y-axis) for the formulation at the time periods of 1, 2, 3, 6, 9 and 12 months.

### DETAILED DESCRIPTION OF THE INVENTION

The present disclosure related to a pharmaceutical formulation comprising an antibody or a fragment thereof that recognises and binds to human HER2.

The term “human HER2” as used herein includes variants, isoforms, and species homologs of human HER2. Accordingly, antibodies of the invention may, in certain cases, cross-react with HER2 from species other than human. In certain embodiments, the antibodies may be completely specific for one or more human HER2 proteins and may not exhibit species or other types of non-human cross-reactivity. The complete amino acid sequence of an exemplary human HER2 has Swiss-Prot accession number P04626 (ERBB2 HUMAN; SEQ ID NO: 1). HER2 is also known as CD340, MLN 19, Neu, c-ErbB-2 and p185erbB2. Human HER2 is designated GeneID: 2064 by Entrez Gene, and HGNC: 3430 by HGNC. HER2 can be encoded by the gene designated ERBB2.

The use of “HER2” herein encompasses all known or as yet undiscovered alleles and polymorphic forms of human HER2. The terms “human HER2” or “HER2” are used herein equivalently and mean “human HER2” if not otherwise specifically indicated.

The terms “antibody that binds to HER2” and “anti-HER2 antibody” are used herein interchangeably and include antibodies or a fragment thereof that bind to human HER2 e.g. human HER2 in isolated form.

The terms “antagonistic antibody” or “antagonist antibody” are used herein equivalently and include an antibody that is capable of inhibiting and/or neutralising the biological signalling activity of HER2, for example by blocking binding or substantially reducing binding of HER2 to a ligand and thus inhibiting or reducing the signalisation pathway triggered by HER2 and/or inhibiting or reducing a HER2-mediated cell response like cell proliferation.

The term “antibody” as referred to herein includes whole antibodies and any antigen binding fragments or single chains thereof. An “antibody” refers to a glycoprotein comprising at least two heavy (H) chains and two light (L) chains inter-connected by disulfide bonds, or an antigen binding fragment thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR) with are hypervariable in sequence and/or involved in antigen recognition and/or usually form structurally defined loops, interspersed with regions that are more conserved, termed framework regions (FR or FW). Each VH and VL is composed of three CDRs and four FWs, arranged from amino-terminus to carboxy-terminus in the following order: FW1, CDR1, FW2, CDR2, FW3, CDR3 and FW4. The amino acid sequences of FW1, FW2, FW3, and FW4 all together constitute the “non-CDR region” or “non-extended CDR region” of VH or VL as referred to herein.

Antibodies are grouped into classes, also referred to as isotypes, as determined genetically by the constant region. Human constant light chains are classified as kappa (CK) and lambda (Cλ) light chains. Heavy chains are classified as mu (μ), delta (δ), gamma (γ), alpha (α), or epsilon (c), and define the antibody's isotype as IgM, IgD, IgG, IgA, and IgE, respectively. Thus, “isotype” as used herein is meant any of the classes and/or subclasses of immunoglobulins defined by the chemical and antigenic characteristics of their constant regions. The known human immunoglobulin isotypes are IgG1 (IGHG1), IgG2 (IGHG2), IgG3 (IGHG3), IgG4 (IGHG4), IgA1 (IGHA1), IgA2 (IGHA2), IgM (IGHM), IgD (IGHD), and IgE (IGHE). The IgG class is the most commonly used for therapeutic purposes. In humans this class comprises subclasses IgG1, IgG2, IgG3 and IgG4.

Antibody fragments include, but are not limited to, (i) the Fab fragment consisting of VL, VH, CL and CH1 domains, including Fab′ and Fab′-SH, (ii) the Fd fragment consisting of the VH and CH1 domains, (iii) the Fv fragment consisting of the VL and VH domains of a single antibody; (iv) the dAb fragment (Ward E S et al., (1989) Nature, 341: 544-546) which consists of a single variable, (v) F(ab′)2 fragments, a bivalent fragment comprising two linked Fab fragments (vi) single chain Fv molecules (scFv), wherein a VH domain and a VL domain are linked by a peptide linker which allows the two domains to associate to form an antigen binding site (Bird R E et al., (1988) Science 242: 423-426; Huston J S et al., (1988) Proc. Natl. Acad. Sci. USA, 85: 5879-83), (vii) bispecific single chain Fv dimers (PCT/US92/09965), (viii) “diabodies” or “triabodies”, multivalent or multispecific fragments constructed by gene fusion (Tomlinson I & Hollinger P (2000) Methods Enzymol. 326: 461-79; WO94/13804; Holliger P et al., (1993) Proc. Natl. Acad. Sci. USA, 90: 6444-48) and (ix) scFv genetically fused to the same or a different antibody (Coloma M J & Morrison S L (1997) Nature Biotechnology, 15(2): 159-163).

Where bispecific antibodies are to be used, these may be conventional bispecific antibodies, which can be manufactured in a variety of ways (Holliger P & Winter G (1993) Current Opinion Biotechnol, 4: 446-449), e.g. prepared chemically or from hybrid hybridomas, or may be any of the bispecific antibody fragments mentioned above. Diabodies and scFv can be constructed without an Fc region using only variable domains, potentially reducing the effects of anti-idiotypic reaction.

The term “chimeric antibody” includes antibodies in which the variable region sequences are derived from one species and the constant region sequences are derived from another species, such as an antibody in which the variable region sequences are derived from a mouse antibody and the constant region sequences are derived from a human antibody.

The term “humanized antibody” or “humanized anti-HER2 antibody” as used herein includes antibodies in which CDR sequences derived from the germline of another mammalian species, such as a mouse, have been grafted onto human framework sequences. Additional framework region modifications may be made within the human framework sequences as well as within the CDR sequences derived from the germline of another mammalian species.

Preferably, the anti-HER2 antibody or a fragment thereof is a monoclonal anti-HER2 antibody, more preferably a humanized anti-HER2 monoclonal antibody. For example, in one particularly preferred embodiment, the anti-HER2 antibody or a fragment thereof has a set of heavy and light CDRs comprising HCDR1-3 (SEQ ID NOS: 2-4) and LCDR1-3 (SEQ ID NOS: 5-7).

The relevant set of CDRs is provided within antibody framework regions. Preferably, antibody framework regions are employed and are preferably germline, more preferably the antibody framework region for the heavy chain may IGHV3-66. The preferred framework region for the light chain may be IGKV1-39. In a preferred embodiment, a VH domain is provided with the amino acid sequence of SEQ ID NO: 8. In a further preferred embodiment, a VL domain is provided with the amino acid sequence of SEQ ID NO: 9 In a highly preferred embodiment, the anti-HER2 antibody comprises a heavy chain and light chain of SEQ ID NO: 10 and SEQ ID NO: 11.

The term “variant antibody” or “antibody variant” as used herein includes an antibody sequence that differs from that of a parent antibody sequence by virtue of at least one amino acid modification compared to the parent. The variant antibody sequence herein will preferably possess at least about 80%, most preferably at least about 90%, more preferably at least about 95% amino acid sequence identity with a parent antibody sequence. Antibody variant may refer to the antibody itself, formulations comprising the antibody variant, or the amino acid sequence that encodes it.

The term “amino acid modification” herein includes an amino acid substitution, insertion, and/or deletion in a polypeptide sequence. By “amino acid substitution” or “substitution” herein is meant the replacement of an amino acid at a particular position in a parent polypeptide sequence with another amino acid. For example, the substitution R94K refers to a variant polypeptide, in this case a heavy chain variable framework region variant, in which the arginine at position 94 is replaced with a lysine. For the preceding example, 94K indicates the substitution of position 94 with a lysine. For the purposes herein, multiple substitutions are typically separated by a slash. For example, R94K/L78V refers to a double variant comprising the substitutions R94K and L78V. By “amino acid insertion” or “insertion” as used herein is meant the addition of an amino acid at a particular position in a parent polypeptide sequence. For example, insert −94 designates an insertion at position 94. By “amino acid deletion” or “deletion” as used herein is meant the removal of an amino acid at a particular position in a parent polypeptide sequence. For example, R94-designates the deletion of arginine at position 94.

As used herein, the term “conservative modifications” or “conservative sequence modifications” is intended to refer to amino acid modifications that do not significantly affect or alter the binding characteristics of the antibody containing the amino acid sequence. Such conservative modifications include amino acid substitutions, insertions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions or within the framework regions of an antibody of the invention can be replaced with other amino acid residues from the same side chain family and the altered antibody (variant antibody) can be tested for retained function.

The term “effector function” as used herein includes a biochemical event that results from the interaction of an antibody Fc region with an Fc receptor or ligand. Effector functions include FcγR-mediated effector functions such as ADCC (antibody dependent cell-mediated cytotoxicity) and ADCP (antibody dependent cell-mediated phagocytosis), and complement-mediated effector functions such as CDC (complement dependent cytotoxicity). An effector function of an antibody may be altered by altering, i.e. enhancing or reducing, preferably enhancing, the affinity of the antibody for an effector molecule such as an Fc receptor or a complement component. Binding affinity will generally be varied by modifying the effector molecule binding site, and in this case it is appropriate to locate the site of interest and modify at least part of the site in a suitable way. It is also envisaged that an alteration in the binding site on the antibody for the effector molecule need not alter significantly the overall binding affinity but may alter the geometry of the interaction rendering the effector mechanism ineffective as in non-productive binding. It is further envisaged that an effector function may also be altered by modifying a site not directly involved in effector molecule binding, but otherwise involved in performance of the effector function. By altering an effector function of an antibody it may be possible to control various aspects of the immune response, e.g. enhancing or suppressing various reactions of the immune system, with possible beneficial effects in diagnosis and therapy.

As used herein, the term “HER2-related disorder” includes conditions such as cancer, particularly metastatic breast cancer, early breast cancer and metastatic gastric cancer and more particularly HER2-positive metastatic breast cancer.

As used herein, the term “subject” includes any human or non-human animal. The term “non-human animal” includes all vertebrates, e.g., mammals and non-mammals, such as non-human primates, sheep, dogs, cats, horses, cows, chickens, amphibians, reptiles, etc. Preferably the subject is human.

A “stable” formulation is one in which the protein therein essentially retains its physical stability and/or chemical stability and/or biological activity upon storage. Various analytical techniques for measuring protein stability are available in the art and are reviewed for example in Peptide and Protein Drug Delivery, 247-301, Vincent Lee Ed., Marcel Dekker, Inc., New York, N.Y., Pubs. (1991) and Jones A (1993) Adv Drug Delivery Rev, 10: 29-90. Stability can be measured at a selected temperature for a selected time period. Preferably, the formulation is stable at room temperature (25° C.) or at 40° C. for at least 1 month, preferably 2 months, more preferably 6 months and/or stable at about 5° C. for at least 1 year and preferably for at least 2 years. Furthermore, the formulation is preferably stable following freezing (to for example, −40° C.) and thawing of the formulation.

The “physical stability” of a protein in a pharmaceutical formulation is retained if it shows no signs of aggregation, precipitation and/or denaturation upon visual examination of colour and/or clarity, or as measured by UV light scattering or by size exclusion chromatography.

The “chemical stability” of a protein can be assessed by detecting and quantifying chemically altered forms of the protein. Chemical alteration may involve size modification (e.g. clipping) which can be evaluated using size exclusion chromatography, SDS-PAGE and/or matrix assisted laser desorption ionization/time-of-flight mass spectrometry (MALDI/TOF MS), for example. Other types of chemical alteration include charge alteration (e.g. occurring as a result of deamidation) which can be evaluated by ion exchange chromatography, for example.

The term “buffer” refers to a buffered solution that resists changes in pH by the action of its acid-base conjugate components. A buffer of this invention has a pH in the range from about 5.0 to about 6.0; and preferably 5.7. Examples of buffers that can control the pH in this range include acetate (e.g. sodium acetate), succinate (such as sodium succinate), gluconate, histidine, citrate and other organic acid buffers.

A “lyoprotectant” is a molecule which when combined with a protein of interest significantly prevents or reduces chemical and/or physical instability of the protein upon lyophilization and subsequence storage. Examples of lyoprotectants include a polyol, an amino acid, a methylamine such as betaine, a lyotropic salt such as magnesium sulphate, propylene glycol, polyethylene glycol, Pluronics, and combinations thereof. The preferred lyoprotectant is a non-reducing sugar such as sucrose and/or a sugar alcohol such as mannitol, more preferably a non-reducing sugar such as sucrose. The lyoprotectant is added to the pre-lyophilisation formulation in a “lyoprotecting amount” which means that following lyophilisation of the protein in the presence of the lyoprotecting amount of the lyoprotectant, the protein retains its physical and chemical stability and integrity upon lyophilization and storage.

A “polyol” is a substance with multiple hydroxyl groups, and includes sugars (reducing and non-reducing sugars), sugar alcohols and sugar acids. Preferred polyols herein have a molecular weight which is less than about 600 kD (e.g. in the range from about 120 to about 400 kD). A “reducing sugar” is one which contains a hemiacetal group that can reduce metal ions or react covalently with lysine and other amino groups in proteins and a “non-reducing sugar” is one which does not have these properties of a reducing sugar. Examples of reducing sugars are fructose, mannose, maltose, lactose, arabinose, xylose, ribose, rhamnose, galactose and glucose. Non-reducing sugars include sucrose, trehalose, sorbose and raffinose. Sugar alcohols include mannitol, xylitol, erythritol, threitol, sorbitol and glycerol. A polyol for use in a formulation that is freeze-thaw stable is one which does not crystallize at freezing temperatures (e.g. −20° C.) such that it destabilises the antibody in the formulation.

A “bulking agent” is a compound which adds mass to the lyophilized mixture and contributes to the physical structure of the lyophilized cake (e.g. facilitates the production of an essentially uniform lyophilized cake which maintains an open pore structure) Examples of bulking agents include mannitol, glycine, polyethylene glycol and xorbitol.

A “liquid” formulation is one that has been prepared in a liquid format. Such a formulation may be suitable for direct administration to a subject or, alternatively, can be packaged for storage either in a liquid form, in a frozen state or in a dried form (e.g. lyophilized) for later reconstitution into a liquid form or other form suitable for administration to a subject.

A “lyophilized” formulation is one that has been prepared by freeze-drying a liquid or pre-lyophilization formulation. Freeze-drying is performed by freezing the formulation and then subliming ice from the frozen content at a temperature suitable for primary drying. Under this condition the product temperature is below the collapse temperature of the formulation. A secondary drying stage may then be carried out, which produces a suitable lyophilized cake.

A “reconstituted” formulation is one that has been prepared by dissolving a lyophilized protein formulation in a diluent such that the protein is dispersed in the reconstituted formulation. The reconstituted formulation should be suitable for administration (e.g. parenteral administration) to a subject to be treated with the protein of interest. Suitable “diluents” useful for the preparation of a reconstituted formulation include ones which are pharmaceutically acceptable (safe and non-toxic for administration to a human). Examples of suitable diluents include sterile water, bacteriostatic water for injection (BWFI). Water for injection (WFI), a pH buffered solution e.g. phosphate-buffered saline (PBS), sterile saline solution, Ringer's solution or dextrose solution.

The antibody comprised by the formulation of the invention can be produced e.g. by recombinant technology. A nucleic acid encoding a HER2 antibody of the invention (e.g. CDR or set of CDRs or VH domain or VL domain or antibody antigen-binding site or antibody molecule, e.g. scFv or IgG1 as provided), may be expressed by culturing under appropriate conditions recombinant host cells containing said nucleic acid. Following production by expression a VH and/or VL domain, for example, may be isolated and/or purified using any suitable technique, then used as appropriate.

Antibodies, VH and/or VL domains, and encoding nucleic acid molecules and vectors may be provided isolated and/or purified, e.g. from their natural environment, in substantially pure or homogeneous form, or, in the case of nucleic acid, free or substantially free of nucleic acid or gene origin other than the sequence encoding a polypeptide with the required function. Nucleic acid may comprise DNA or RNA and may be wholly or partially synthetic.

Systems for cloning and expression of a polypeptide in a variety of different host cells are well known. Suitable host cells include bacteria, mammalian cells, plant cells, yeast and baculovirus systems and transgenic plants and animals.

Mammalian cell lines available in the art for expression of a heterologous polypeptide include Chinese hamster ovary (CHO) cells, HeLa cells, baby hamster kidney cells, NSO mouse melanoma cells, YB2/0 rat myeloma cells, human embryonic kidney cells, human embryonic retina cells and many others.

Preferably, the mammalian cell line is Chinese hamster ovary (CHO) cells. These may be dihydrofolate reductase (dhfr) deficient and so dependent on thymidine and hypoxanthine for growth (Urlaub G & Chasin L A (1980) PNAS, 77: 4216-4220). The parental dhfr CHO cell line is transfected with the antibody gene and dhfr gene which enables selection of CHO cell transformants of dhfr positive phenotype. Selection is carried out by culturing the colonies on media devoid of thymidine and hypoxanthine, the absence of which prevents untransformed cells from growing and transformed cells from resalvaging the folate pathway and thereby bypassing the selection system. These transformants usually express low levels of the product gene by virtue of co-integration of both transfected genes. The expression levels of the antibody gene may be increased by amplification using methotrexate (MTX). This drug is a direct inhibitor of the dhfr enzyme and allows isolation of resistant colonies which amplify their dhfr gene copy number sufficiently to survive under these conditions. Since the dhfr and antibody genes are more closely linked in the original transformants, there is usually concomitant amplification, and therefore increased expression of the desired antibody gene. Another selection system for use with CHO or myeloma cells is the glutamine synthetase (GS) amplification system described in WO87/04462. This system involves the transfection of a cell with a gene encoding the GS enzyme and the desired antibody gene. Cells are then selected which grow in glutamine free medium. These selected clones are then subjected to inhibition of the GS enzyme using methionine sulphoximine (MSX). The cells, in order to survive, will amplify the GS gene with concomitant amplification of the gene encoding the antibody of interest.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. ‘phage, or phagemid, as appropriate. For further details see, for example, Molecular Cloning: a Laboratory Manual: 3rd edition, Sambrook and Russell, 2001, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1988, Short Protocols in Molecular Biology; A Compendium of Methods from Current Protocols in Molecular Biology, Ausubel et al. eds., John Wiley & Sons, 4th edition 1999. The disclosures of Sambrook et al. and Ausubel et al. (both) are incorporated herein by reference.

Introduction of a nucleic acid into a host cell may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. Introducing nucleic acid in the host cell, in particular a eukaryotic cell may use a viral or a plasmid based system. The plasmid system may be maintained episomally or may be incorporated into the host cell or into an artificial chromosome (Csonka E et al., (2000) Journal of Cell Science, 113: 3207-3216; Vanderbyl S et al., (2002) Molecular Therapy, 5(5): 10. Incorporation may be either by random or targeted integration of one or more copies at single or multiple loci. For bacterial cells, suitable techniques may include calcium chloride transformation, electroporation and infection using bacteriophage.

The introduction may be followed by causing or allowing expression from the nucleic acid, e.g. by culturing host cells under conditions for expression of the gene. In one embodiment, the nucleic acid of the invention is integrated into the genome (e. g. chromosome) of the host cell. Integration may be promoted by inclusion of sequences which promote recombination with the genome, in accordance with standard techniques.

A further embodiment provides a process for purifying a HER2 antibody which comprises one or more chromatographic separation steps wherein each of said separation steps comprises elution with an elution buffer comprising one or more pharmaceutically acceptable excipients.

Preferably, the one or more chromatographic separation steps are selected from affinity chromatography (e.g. Protein A or Protein G affinity chromatography), ion exchange chromatography (e.g. cation and anion exchange chromatography), hydrophobic interaction chromatography (e.g. phenyl chromatography), hydroxy apatite chromatography, size exclusion chromatography, immobilised metal affinity chromatography, hydrophilic interaction chromatography, thiophilic adsorption chromatography, euglobulin adsorption chromatography, dye ligand chromatography or immobilised boronate chromatography. Most preferably, chromatographic separation is performed by Protein A affinity chromatography followed by cation exchange chromatography and/or followed by hydrophobic interaction chromatography or anion exchange chromatography.

### Pharmaceutical Formulations

The pharmaceutical formulation of the invention may be a liquid formulation, a lyophilized formulation or a reconstituted formulation.

The desired dose volume and ultimate mode of administration of the formulation is taken into account when determining the amount of anti-HER2 antibody or a fragment thereof to include in the formulation. Preferably the anti-HER2 antibody of a fragment thereof is present within the pharmaceutical formulation in an amount of between 1 mg/ml and 100 mg/ml, more preferably 5 mg/ml and 50 mg/ml, even more preferably 10 mg/ml and 40 mg/ml, especially 20 mg/ml and 30 mg/ml.

The formulation can be buffered to a pH of 5.0-6.0, preferably pH 5.5-5.9, more preferably pH 5.6-5.8, even more preferably pH 5.7±0.2, most preferably pH 5.7±0.1. Examples of buffers that can be used to control the pH in this range include acetate, citrate, succinate, gluconate, histidine, phosphate, glutaric, cacodylyte, sodium hydrogen maleate, tris-(hydroxylmethyl) aminomethane (Tris), 2-(N-morpholino) ethanesulphonic acid (MES), imidazole and other organic acid buffers. Preferably, the buffer is acetate buffer, more preferably sodium acetate. Preferably, the acetate buffer is present within the formulation in an amount of between 1-50 mM, more preferably 1-20 mM, even more preferably 1-10 mM.

Preferably, a “pharmaceutically acceptable excipient” is added to the liquid formulation. It will be appreciated that references to “pharmaceutically acceptable excipient” includes references to any excipient conventionally used in pharmaceutical formulations. Such excipients may typically include one or more surfactant, lyoprotectant, bulking agent, inorganic or organic salt, stabilizer, diluent, solubilizer, reducing agent, antioxidant, chelating agent, preservative and the like.

Since it is desirable for a parenteral formulation to be isotonic with body fluids (i.e. approximately 284 mOsm/L) a tonicifier may be added to the pharmaceutical formulation. An example of a commonly used tonicifier is sodium chloride salt. Preferably, the tonicifier is present in the liquid formulation at a concentration of 50 to 400 mM, more preferably 100 to 300 mM, even more preferably 200 to 250 mM. However a tonicifier is not usually added to a lyophilized formulation, therefore prior to lyophilisation, a tonicifier may be removed from the formulation. A tonicifier may be present in the diluent for reconstituted formulations.

Examples of a typical surfactant include: non-ionic surfactants (HLB 6 to 18) such as sorbitan fatty acid esters (e.g. sorbitan monocaprylate, sorbitan monolaurate, sorbitan monopalmitate), glycerine fatty acid esters (e.g. glycerine monocaprylate, glycerine monomyristate, glycerine monostearate), poly glycerine fatty acid esters (e.g. decaglyceryl monostearate, decaglyceryl distearate, decaglyceryl monolinoleate), polyoxyethylene sorbitan fatty acid esters (e.g. polyoxyethylene sorbitan monolaurate, polyoxyethylene sorbitan monooleate, polyoxyethylene sorbitan monostearate, polyoxyethylene sorbitan monopalmitate, polyoxyethylene sorbitan trioleate, polyoxyethylene sorbitan tristearate), polyoxyethylene sorbitol fatty acid esters (e.g. polyoxyethylene sorbitol tetrastearate, polyoxyethylene sorbitol tetraoleate), polyoxyethylene glycerine fatty acid esters (e.g. polyoxyethylene glyceryl monostearate), polyethylene glycol fatty acid esters (e.g. polyethylene glycol distearate), polyoxyethylene alkyl ethers (e.g. polyoxyethylene lauryl ether), polyoxyethylene polyoxypropylene alkyl ethers (e.g. polyoxyethylene polyoxypropylene glycol ether, polyoxyethylene polyoxypropylene propyl ether, polyoxyethylene polyoxypropylene cetyl ether), polyoxyethylene alkylphenyl ethers (e.g. polyoxyethylene nonylphenyl ether), polyoxyethylene hydrogenated castor oils (e.g. polyoxyethylene castor oil, polyoxyethylene hydrogenated castor oil), polyoxyethylene beeswax derivatives (e.g. polyoxyethylene sorbitol beeswax), polyoxyethylene lanolin derivatives (e.g. polyoxyethylene lanolin), and polyoxyethylene fatty acid amides (e.g. polyoxyethylene stearyl amide); anionic surfactants such as C₁₀-C₁₈ alkyl sulfates salts (e.g. sodium cetyl sulfate, sodium lauryl sulfate, sodium oleyl sulfate), polyoxyethylene C₁₀-C₁₈ alkyl ether sulfates salts with an average of 2-4 moles of ethylene oxide (e.g. sodium polyoxyethylene lauryl sulfate), and C₈-C₁₈ alkyl sulfosuccinate ester salts (e.g. sodium lauryl sulfosuccinate ester); and natural surfactants such as lecithin, glycerophospholipid, sphingophospholipids (e.g. sphingomyelin) and sucrose esters of C₁₂-C₁₈ fatty acids.

Preferably, the surfactant is selected from polyoxyethylene sorbitan fatty acid esters. Particularly preferably the surfactant is Polysorbate 20, 21, 40, 60, 65, 80, 81 and 85, most preferably Polysorbate 80. Polysorbate 80 is also known by the brand name Tween 80™ (ICI Americas, Inc.).

Preferably, the surfactant is present within the formulation in an amount of between 0.001 and 0.1% (w/w), more preferably between 0.001 and 0.05% (w/w), even more preferably between 0.005 and 0.02% (w/w).

A solubilizer may also be added to increase the solubilisation of the antibody in solution. Examples of solubilizers include amino acids such as proline or glycine, prophylene glycol, plasdone C and K povidones, cyclodextrins and plasdone K polymers. Preferably, the solubilizer is added to the formulation at a concentration of 1-50 mg/ml, preferably 5-40 mg/ml, more preferably 10-30 mg/ml.

Pharmaceutical formulations of the invention may also comprise additional excipients such as reducing agents, antioxidants and/or chelating agents.

Examples of a reducing agent include N-acetylcysteine, N-acetylhomocysteine, thioctic acid, thiodiglycol, thioethanolamine, thioglycerol, thiosorbitol, thioglycolic acid and a salt thereof, sodium thiosulfate, glutathione and a C₁-C₇ thioalkanoic acid.

Examples of an antioxidant include amino acids such as methionine, erythorbic acid, dibutylhydroxytoluene, butylhydroxyanisole, alpha-tocopherol, tocopherol acetate, L-ascorbic acid and a salt thereof, L-ascorbic acid palmitate, L-ascorbic acid stearate, sodium bisulfite, sodium sulfite, triamyl gallate and propyl gallate.

Examples of a chelating agent include disodium ethylenediaminetetraacetate (EDTA), sodium pyrophosphate and sodium metaphosphate.

Not all excipients in a liquid formulation are suitable for inclusion a lyophilized form of the formulation and therefore a number of changes may be made to the liquid formulation to make it suitable for freeze-drying.

A stabilizer may be added to the formulation to stabilise the protein in the lyophilised form. Examples of a stabiliser include creatinine, an amino acid selected from histidine, alanine, glutamic acid, glycine, leucine, phenylalanine, methionine, isoleucine, proline, aspartic acid, arginine, lysine and threonine, a carbohydrate selected from sucrose, trehalose, sorbitol, xylitol and mannose, surfactants selected from polyethylene glycol (PEG; e.g. PEG3350 or PEG4000) or polyoxyethylene sorbitan fatty acid esters (e.g. Polysorbate 20 or Polysorbate 80), or any combination thereof.

In a preferred embodiment the stabiliser comprises a lyoprotectant, which may be selected from a non-reducing sugar (e.g. sucrose or trehalose) and/or a sugar alcohol (e.g. mannitol). Addition of a lyoprotectant helps to reduce the amount of degradation or aggregation of the protein upon lyophilisation. Preferably, the lyophilized formulation is isotonic upon reconstitution; therefore the amount of lyoprotectant in the lyophilized formulation should be sufficient to achieve an isotonic reconstituted formulation. Alternatively, the reconstituted formulation may be hypertonic and therefore a greater amount of lyoprotectant is required in the lyophilized formulation. Conversely, if too little lyoprotectant is added to the lyophilized formulation, then an unacceptable amount of antibody degradation or aggregation may occur upon lyophilisation.

Preferably, the lyoprotectant in the lyophilized formulation is a non-reducing sugar and/or a sugar alcohol, more preferably sucrose and/or mannitol. In preferred embodiment the lyoprotectant in the lyophilized formulation is sucrose. Preferably the lyoprotectant is present in the lyophilized formulation at a concentration from about 10 mM to about 700 mM, preferably from about 50 mM to about 600 mM, more preferably from about 100 mM to about 500 mM, even more preferably from about 200 mM to about 400 mM.

Preferably, the lyoprotectant is present in the lyophilized formulation in an amount of between 1-100% (w/w), preferably 2-50% (w/w), more preferably 3-25%, even more preferably 5-20% (w/w).

In a lyophilized formulation comprising a lyoprotectant, preferably the molar ratio of antibody to lyoprotectant may be in the range from 100 to about 3000 moles lyoprotectant to 1 mole antibody, preferably from about 500 to about 2500 moles lyoprotectant to 1 mole antibody, more preferably from about 1000 to about 2250 moles lyoprotectant to 1 mole antibody, even more preferably from about 1500 to 2000 moles.

Preferably, the lyophilized formulation comprises a bulking agent. Examples of bulking agents include mannitol, glycine, polyethylene glycol, xorbitol, anhydrous lactose, sucrose, D(+)-trehalose, dextran 40 and povidone (PVP K24). Preferably, the bulking agent is present in the lyophilized formulation at a concentration from about 50 to 200 mM, preferably from about 100 to 150 mM. Preferably, the bulking agent is present in the lyophilized formulation in an amount of between 0.1 and 10% (w/w), preferably between 0.2 and 8% (w/w), more preferably between 0.5 and 5% (w/w).

Once a lyophilized formulation has been prepared, the material is subjected to freeze-drying. This can be achieved using a number of commercially available freeze-dryers. These work by freezing the material and then reducing the surrounding pressure to allow the frozen water in the material to sublimate directly from the solid phase to the gas phase. A secondary drying phase may also be carried out. Preferably the lyophilized formulation comprises a moisture content of less than 5%, preferably less than 4%, more preferably less than 3%, even more preferably less than 2%.

The lyophilized formulation may be reconstituted with a diluent such that the antibody concentration in the reconstituted formulation is present in an amount of between 1 and 100 mg/ml, more preferably 1 and 50 mg/ml.

Diluents for use in reconstitution of the lyophilized formulation include sterile water, bacteriostatic water for injection (BWFI), water for injection (WFI) a pH buffered solution such as phosphate buffered saline, sterile saline solution, Ringer's solution or dextrose solution.

A preservative may be added to the diluent to reduce bacterial action in the reconstituted formulation Examples of a preservative include octadecyldimethylbenzyl ammonium chloride, hexamethonium chloride, benzalkonium chloride (a mixture of alkylbenzyldimethyl-ammonium chlorides in which the alkyl groups are long-chain compounds), benzethonium chloride, aromatic alcohols such as phenol, butyl and benzyl alcohol, alkyl parabens such as methyl or propyl paraben, catechol, resorcinol, cyclohexanol, 3-pentanol, and m-cresol.

In an aspect of the present invention, a pharmaceutical formulation is produced as a liquid formulation. Preferably the anti-HER2 antibody or a fragment thereof is present within the liquid formulation in an amount of between 1 mg/ml and 100 mg/ml, preferably 5 mg/ml and 50 mg/ml, preferably 10 mg/ml and 40 mg/ml, more preferably 15 mg/ml and 30 mg/ml, even more preferably 20-25 mg/ml, especially 21 mg/ml.

The liquid formulation can be buffered to a pH of 5.0-6.0, preferably pH 5.5-5.9, more preferably pH 5.6-5.8, even more preferably pH 5.7±0.2, most preferably pH 5.7±0.1, especially pH 5.7. As detailed in Example 1, the applicants have found that an acetate buffer confers significant stability to the liquid formulation, compared to, for example, a citrate buffer. Therefore, the preferred buffer is acetate buffer, more preferably sodium acetate. Preferably, the acetate buffer is present within the liquid formulation in an amount of between 1-50 mM, preferably 1-20 mM, more preferably 1-10 mM, even more preferably 2-7 mM, especially 4 mM.

Preferably, a tonicifier is present in the liquid formulation at a concentration of 50 to 400 mM, preferably 100 to 300 mM, more preferably 200 to 250 mM, even more preferably 210-220 mM, especially 214 mM.

Preferably, a surfactant is added to the liquid formulation, which may be selected from polyoxyethylene sorbitan fatty acid esters. Particularly preferably the surfactant is Polysorbate 80 (also known by the brand name Tween 80™ (ICI Americas, Inc.)).

Preferably, the surfactant is present within the liquid formulation in an amount of between 0.001 and 0.1% (w/w), more preferably between 0.002 and 0.05% (w/w), even more preferably between 0.005 and 0.02% (w/w) and especially 0.01% (w/w).

Preferably a solubilizer is added to the liquid formulation. The solubilizer may be proline or glycine, preferably proline. Preferably, the solubilizer is added to the formulation at a concentration of 1-50 mg/ml, preferably 5-40 mg/ml, more preferably 10-30 mg/ml, even more preferably 15-25 mg/ml, most preferably at 20 mg/ml.

In a preferred embodiment of the invention, the liquid formulation comprises an anti-HER2 antibody or a fragment thereof and pharmaceutically acceptable excipients, buffered to a pH of 5.7±0.2 with acetate buffer. In a further preferred embodiment of the invention, the liquid formulation comprises an anti-HER2 antibody or a fragment thereof formulated with NaCl, proline, Polysorbate 80 and sodium acetate, at a pH of 5.7. Preferably the anti-HER2 antibody or a fragment thereof comprises a heavy chain and light chain of SEQ ID NO: 10 and SEQ ID NO: 11.

In a yet further preferred embodiment of the invention, the liquid formulation comprises 10-40 mg/ml of an anti-HER2 antibody or a fragment thereof, 200-250 mM NaCl, 10-30 mg/ml proline, 0.005 and 0.02% (w/w) Polysorbate 80 and 1-10 mM sodium acetate buffer, wherein the pH of the formulation is pH 5.7±0.2. Preferably, the anti-HER2 antibody or a fragment thereof comprises a heavy chain and light chain of SEQ ID NO: 10 and SEQ ID NO: 11.

In a more preferred embodiment of the invention, the liquid formulation comprises 21 mg/ml of an anti-HER2 antibody or a fragment thereof, 214 mM NaCl, 20 mg/ml proline, 0.01% (w/w) Polysorbate 80 and 4 mM sodium acetate buffer, wherein the pH of the formulation is pH5.7. Preferably, the anti-HER2 antibody or a fragment thereof comprises a heavy chain and light chain of SEQ ID NO: 10 and SEQ ID NO: 11.

In a further aspect of the present invention, the pharmaceutical formulation comprises a lyophilized formulation of an antibody.

Preferably the anti-HER2 antibody or a fragment thereof is present within the lyophilized formulation in an amount of between 1 mg/ml and 100 mg/ml, preferably 5 mg/ml and 80 mg/ml, preferably 10 mg/ml and 60 mg/ml, preferably 15 mg/ml and 40 mg/ml, more preferably 20-40 mg/ml, even more preferably 25-35 mg/ml, especially 30 mg/ml.

The lyophilized formulation can be buffered to a pH of 5.0-6.0, preferably pH 5.5-5.9, more preferably pH 5.6-5.8, even more preferably pH 5.7±0.2, most preferably pH 5.7±0.1, especially pH 5.7. The preferred buffer is acetate buffer, more preferably sodium acetate. Preferably, the acetate buffer is present within the lyophilized formulation in an amount of between 1-50 mM, preferably 1-20 mM, more preferably 1-10 mM, even more preferably 3-7 mM, especially 5-6 mM.

In a preferred embodiment, the lyophilized formulation comprises a stabiliser. Preferably, the stabiliser is a lyoprotectant, which is a non-reducing sugar and/or a sugar alcohol. Preferably the lyoprotectant is sucrose and/or mannitol, most preferably sucrose. Preferably the lyoprotectant is present in the lyophilized formulation at a concentration from about 50 mM to about 700 mM, preferably from about 100 mM to about 600 mM, more preferably from about 200 mM to about 500 mM, even more preferably from about 300 mM to about 400 mM and most preferably at about 350 mM.

In a lyophilized formulation comprising sucrose as the lyoprotectant, preferably sucrose is present in the lyophilized formulation in an amount of between 1-90% (w/w), preferably 2-50% (w/w), preferably 4-25% (w/w), more preferably 7-20% (w/w), even more preferably 10-15% (w/w) and most preferably at an amount of 12% (w/w).

In a lyophilized formulation comprising mannitol as the lyoprotectant, preferably mannitol is present in the lyophilized formulation in an amount of between 0.1-20% (w/w), preferably, 0.5-10%, more preferably 1-6%, even more preferably 2-4%, most preferably 2%.

In a lyophilized formulation comprising sucrose as the lyoprotectant, preferably the molar ratio of anti-HER2 antibody to lyoprotectant may be in the range from 100 to about 3000 moles lyoprotectant to 1 mole antibody, preferably from about 500 to about 2500 moles lyoprotectant to 1 mole antibody, more preferably from about 1000 to about 2250 moles lyoprotectant to 1 mole antibody, even more preferably from about 1500 to 2000 moles. Most preferably the molar ratio of anti-HER2 antibody to lyoprotectant may be about 1750 moles lyoprotectant to 1 mole antibody.

Preferably, the lyophilized formulation comprises a bulking agent. Preferably, the bulking agent is glycine present in the lyophilized formulation at a concentration from about 50 to 200 mM, preferably from about 100 to 150 mM, more preferably from about 120 to 140 mM, even more preferably from about 130 mM to 135 mM. Preferably, the bulking agent is present in the lyophilized formulation in an amount of between 0.1 and 10% (w/w), preferably between 0.2 and 8% (w/w), more preferably between 0.5 and 5% (w/w), even more preferably between 0.7 and 3% (w/w) and most preferably at 1% (w/w).

Preferably, the lyophilized formulation comprises a surfactant, which is selected from polyoxyethylene sorbitan fatty acid esters. Preferably the surfactant is Polysorbate 80 (also known by the brand name Tween 80™ (ICI Americas, Inc.)). Preferably, the surfactant is present within the lyophilized formulation in an amount of between 0.001 and 0.1% (w/w), more preferably between 0.002 and 0.05% (w/w), even more preferably between 0.005 and 0.02% (w/w) and especially 0.014% (w/w).

In a preferred embodiment of the invention, the lyophilized formulation comprises an anti-HER2 antibody or a fragment thereof, stabilisers and a surfactant, buffered to a pH of 5.7±0.2 with acetate buffer. In a further preferred embodiment of the invention, the lyophilized formulation comprises an anti-HER2 antibody or a fragment thereof, glycine, sucrose, Polysorbate 80 and sodium acetate, at a pH of 5.7±0.2. Preferably the anti-HER2 antibody or a fragment thereof comprises a heavy chain and light chain of SEQ ID NO: 10 and SEQ ID NO: 11.

In a further preferred embodiment of the invention, the lyophilized formulation comprises 20-40 mg/ml of an anti-HER2 antibody or a fragment thereof, 10-15% (w/w) sucrose, 0.5-5% (w/w) glycine, 0.005-0.02% (w/w) Polysorbate 80 and 1-10 mM sodium acetate, wherein the pH of the formulation is pH 5.7±0.2. Preferably the anti-HER2 antibody or a fragment thereof comprises a heavy chain and light chain of SEQ ID NO: 10 and SEQ ID NO: 11.

In a further more preferred embodiment of the invention, the lyophilized formulation comprises 30 mg/ml of an anti-HER2 antibody or a fragment thereof, 12% (w/w) sucrose, 1% (w/w) glycine, 0.014% (w/w) Polysorbate 80 and 5.75 mM sodium acetate, wherein the pH of the formulation is pH 5.7. Preferably the anti-HER2 antibody or a fragment thereof comprises a heavy chain and light chain of SEQ ID NO: 10 and SEQ ID NO: 11.

In a yet further preferred embodiment of the invention, the lyophilized formulation comprises 20-40 mg/ml of an anti-HER2 antibody or a fragment thereof, 300-400 mM sucrose, 120-140 mM glycine, 0.005-0.02% (w/w) Polysorbate 80 and 1-10 mM sodium acetate, wherein the pH of the formulation is pH 5.7±0.2. Preferably the anti-HER2 antibody or a fragment thereof comprises a heavy chain and light chain of SEQ ID NO: 10 and SEQ ID NO: 11.

In a yet further more preferred embodiment of the invention, the lyophilized formulation comprises 30 mg/ml of an anti-HER2 antibody or a fragment thereof, 350 mM sucrose, 133 mM glycine, 0.014% (w/w) Polysorbate 80 and 5.75 mM sodium acetate, wherein the pH of the formulation is pH 5.7. Preferably the anti-HER2 antibody or a fragment thereof comprises a heavy chain and light chain of SEQ ID NO: 10 and SEQ ID NO: 11.

Once the lyophilized formulation has been prepared, the material is subjected to freeze-drying. Preferably the formulation following lyophilization comprises a moisture content of less than 5%, preferably less than 4%, more preferably less than 3%, even more preferably less than 2% and especially between 1% and 2%.

In a further aspect of the present invention, the pharmaceutical formulation comprises a reconstituted formulation of an antibody.

The lyophilized formulation may be reconstituted with a diluent such that the concentration of the anti-HER2 antibody or a fragment thereof in the reconstituted formulation is present in an amount of between 1 mg/ml and 100 mg/ml, preferably 5 mg/ml and 50 mg/ml, preferably 10 mg/ml and 40 mg/ml, more preferably 15 mg/ml and 30 mg/ml, even more preferably 20 and 25 mg/ml, especially 21 mg/ml.

Diluents for use in reconstitution of the lyophilized formulation include sterile water, bacteriostatic water for injection (BWFI), water for injection (WFI), a pH buffered solution such as phosphate buffered saline, sterile saline solution, Ringer's solution or dextrose solution. Preferably, the diluent for use in reconstitution of the lyophilized formulation of the present invention is WFI.

The diluent may be added in an amount of between 0.1-100 ml, preferably between 1-50 ml, more preferably between 2-30 ml, even more preferably between 3-15 ml, especially between 5-10 ml, to yield an antibody solution at the desired concentration. Preferably, the lyophilized formulation is reconstituted to yield a protein concentration of 21 mg/ml with 7.2 ml of WFI.

Preferably, the reconstituted formulation is buffered to a pH of 5.0-6.0, more preferably pH 5.7±0.2, even more preferably 5.7±0.1, most preferably pH 5.7. Preferably, the buffer is acetate buffer, more preferably sodium acetate. Preferably, sodium acetate is present within the reconstituted formulation in an amount of between 1-50 mM, preferably 1-20 mM, more preferably 1-10 mM, even more preferably 2-6 mM, especially 4 mM.

Preferably, the reconstituted formulation comprises a surfactant, which is selected from polyoxyethylene sorbitan fatty acid esters. Preferably the surfactant is Polysorbate 80 (also known by the brand name Tween 80™ (ICI Americas, Inc.)). Preferably, the surfactant is present within the reconstituted formulation in an amount of between 0.001 and 0.1% (w/w), more preferably between 0.002 and 0.05% (w/w), even more preferably between 0.005 and 0.02% (w/w) and especially 0.01% (w/w).

In a preferred embodiment, the reconstituted formulation comprises a stabiliser. Preferably, the stabiliser is a lyoprotectant, which is a non-reducing sugar and/or a sugar alcohol. Preferably the lyoprotectant is sucrose and/or mannitol, most preferably sucrose. Preferably the lyoprotectant is present in the reconstituted formulation at a concentration from about 10 mM to about 700 mM, preferably from about 50 mM to about 600 mM, more preferably from about 100 mM to about 500 mM, even more preferably from about 200 mM to about 300 mM and most preferably at about 225 mM to about 275 mM, especially about 250 mM. Preferably, the lyoprotectant is sucrose, present in the reconstituted formulation in an amount of between 1-50% (w/w), preferably 2-25% (w/w), preferably 4-20% (w/w), more preferably 5-15% (w/w), even more preferably 6-10% (w/w) and most preferably at an amount of 8.4% (w/w).

In a reconstituted formulation comprising mannitol as the lyoprotectant, preferably mannitol is present in the lyophilized formulation in an amount of between 0.1-20% (w/w), preferably, 0.5-10%, more preferably 1-6%, even more preferably 2-4%, most preferably 2%.

In the reconstituted formulation comprising sucrose as the lyoprotectant, preferably the molar ratio of anti-HER2 antibody to lyoprotectant may be in the range from 100 to about 3000 moles lyoprotectant to 1 mole antibody, preferably from about 500 to about 2500 moles lyoprotectant to 1 mole antibody, more preferably from about 1000 to about 2000 moles lyoprotectant to 1 mole antibody. Most preferably the molar ratio of anti-HER2 antibody to lyoprotectant may be about 1750 moles lyoprotectant to 1 mole antibody.

Preferably, the reconstituted formulation comprises a bulking agent. Preferably, the bulking agent is glycine present in the reconstituted formulation at a concentration from about 50 to 200 mM, preferably from about 60 to 150 mM, more preferably from about 70 to 120 mM, even more preferably from about 80 mM to 100 mM, most preferably at about 90 mM. Preferably, the bulking agent is present in the lyophilized formulation in an amount of between 0.1 and 10% (w/w), preferably between 0.2 and 5% (w/w), more preferably between 0.5 and 2% (w/w), even more preferably at 0.7% (w/w).

In a preferred embodiment of the invention, the reconstituted lyophilized formulation comprises an anti-HER2 antibody or a fragment thereof, stabilisers, a surfactant and a diluent, buffered to a pH of 5.7±0.2 with acetate buffer. In a further preferred embodiment of the invention, the reconstituted lyophilized formulation comprises an anti-HER2 antibody or a fragment thereof, glycine, sucrose, Polysorbate 80, WFI and sodium acetate at a pH of 5.7±0.2. Preferably the anti-HER2 antibody or a fragment thereof comprises a heavy chain and light chain of SEQ ID NO: 10 and SEQ ID NO: 11.

In a preferred embodiment of the invention, the reconstituted lyophilized formulation comprises 15-30 mg/ml of an anti-HER2 antibody or a fragment thereof, 5-15% (w/w) sucrose, 0.5-2% (w/w) glycine, 0.005-0.02% (w/w) Polysorbate 80, WFI and 1-10 mM sodium acetate, wherein the pH of the formulation is pH 5.7±0.2. Preferably, the anti-HER2 antibody or a fragment thereof comprises a heavy chain and light chain of SEQ ID NO: 10 and SEQ ID NO: 11.

In a more preferred embodiment of the invention, the reconstituted lyophilized formulation comprises 21 mg/ml of an anti-HER2 antibody or a fragment thereof, 8.4% (w/w) sucrose, 0.7% (w/w) glycine, 0.01% (w/w) Polysorbate 80, WFI and 4 mM sodium acetate, wherein the pH of the formulation is pH 5.7. Preferably, the anti-HER2 antibody or a fragment thereof comprises a heavy chain and light chain of SEQ ID NO: 10 and SEQ ID NO: 11.

In a further preferred embodiment of the invention the reconstituted lyophilized formulation comprises 15-30 mg/ml of an anti-HER2 antibody or a fragment thereof, 225-275 mM sucrose, 80-100 mM glycine, 0.005-0.02% (w/w) Polysorbate 80, 5-10 ml WFI and 1-10 mM sodium acetate buffer, wherein the pH of the formulation is pH 5.7±0.2. Preferably, the anti-HER2 antibody or a fragment thereof comprises a heavy chain and light chain of SEQ ID NO: 10 and SEQ ID NO: 11.

In a further more preferred embodiment of the invention the reconstituted lyophilized formulation comprises 21 mg/ml of an anti-HER2 antibody or a fragment thereof, 246 mM sucrose, 90 mM glycine, 0.01% (w/w) Polysorbate 80, 7.2 ml WFI and 4 mM sodium acetate buffer, wherein the pH of the formulation is pH 5.7. Preferably, the anti-HER2 antibody or a fragment thereof comprises a heavy chain and light chain of SEQ ID NO: 10 and SEQ ID NO: 11.

According to a further aspect of the invention there is provided a use of a pharmaceutical antibody formulation as defined herein for the treatment of a HER2 related disorder.

Preferably, the HER2 related disorder is selected from cancer. Examples of cancer to be treated herein include, but are not limited to, carcinoma, lymphoma, blastoma, sarcoma, and leukemia or lymphoid malignancies. Particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. More particular examples include a HER2 positive cancer such as metastatic breast cancer, early breast cancer and metastatic gastric cancer. Most preferably, the HER2 related disorder is HER2 positive metastatic breast cancer.

The invention further provides a method of treatment or prophylaxis of a HER2 related disorder which comprises administering to a subject a therapeutically effective amount of a pharmaceutical antibody formulation as defined herein.

The invention further provides a pharmaceutical antibody formulation as defined herein for use in the treatment of a HER2 related disorder.

The pharmaceutical formulation of the invention may be a liquid formulation, a lyophilized formulation or a reconstituted formulation i.e. a lyophilized formulation which is reconstituted before use. A liquid formulation is usually provided in the form of containers with defined volume, including sealed and sterilized plastic or glass vials, ampoules and syringes, as well as in the form of large volume containers like bottles. A lyophilized formulation is usually provided in the form of a powder of a defined weight in sealed and sterilized plastic or glass vials. The powder is reconstituted before use. Preferably, the pharmaceutical formulation of the invention is a lyophilized formulation. More preferably the pharmaceutical formulation of the invention is a reconstituted formulation. The pharmaceutical formulation of the invention may be administered orally, or by injection (for example, subcutaneously, intravenously, intraperitoneal or intramuscularly), or by inhalation or topically (for example intraocular, intranasal, rectal, into wounds, on skin). The route of administration can be determined by the physicochemical characteristics of the treatment, by special considerations for the disease or by the requirement to optimise efficacy or to minimise side-effects. Preferably, the formulation of the invention is administered by intravenous infusion, e.g., as a bolus and/or by continuous infusion over a period of time.

An embodiment of the invention may comprise an article of manufacture comprising:

(a) a container which holds a lyophilized formulation of an anti-HER2 antibody or a fragment thereof, a lyoprotectant and an acetate buffer; and
(b) instructions for reconstituting the lyophilized formulation with a diluent to an antibody concentration in the reconstituted formulation of about 10-40 mg/ml.

Preferably, the lyophilized formulation is reconstituted with a diluent to yield an antibody concentration of about 12-30 mg/ml. More preferably, the antibody concentration is about 15-25 mg/ml, even more preferably about 21 mg/ml.

In accordance with the invention, formulations provided may be administered to individuals. Administration is preferably in a “therapeutically effective amount”, this being sufficient to show benefit to a subject. Such benefit may be at least amelioration of at least one symptom. The actual amount administered, and rate and time-course of administration, will depend on the nature and severity of what is being treated. Prescription of treatment, e.g. decisions on dosage etc, is within the responsibility of medical doctors. Appropriate doses of antibody are well known in the art (Ledermann J A et al., (1999) Int J Cancer 47: 659-664; Bagshawe K D et al., (1991) Antibody, Immunoconjugates and Radiopharmaceuticals, 4: 915-922). The precise dose will depend upon a number of factors, including the size and location of the area to be treated, body weight of the subject, the precise nature of the antibody (e.g. whole antibody or fragment) and any additional therapeutic agents administered before, at the time of or after administration of the antibody. A typical antibody dose will be in the range 2 mg/kg to 8 mg/kg for intravenous administration.

The antibody or a fragment thereof is suitably administered to the subject at one time or over a series of treatments. Depending on the type and severity of the disease, about 0.1 mg/kg to 15 mg/kg of antibody is an initial candidate dosage for administration to the subject, for example, by one or more separate administrations, or by continuous infusion. A typical daily dosage might range from about 0.1 mg/kg to 50 mg/kg or more, depending on the factors mentioned above. For repeated administrations over several days or longer, depending on the condition, the treatment is sustained until a desired suppression of disease symptoms occurs. The preferred dosage of the antibody will be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the subject. Such doses may be administered intermittently, e.g. every week or every three weeks (e.g. such that the subject receives from about two to about 20, e.g. about six doses of the anti-HER2 antibody). An initial higher loading dose, followed by one or more lower doses may be administered. An exemplary dosing regimen comprises administering an initial loading dose of about 4 mg/kg to 8 mg/kg, followed by a weekly maintenance dose of about 2 mg/kg to 6 mg/kg of the anti-HER2 antibody. Preferably the initial loading dose is 8 mg/kg followed by a weekly or three weekly dose of 2 mg/kg or 6 mg/kg, respectively.

Other therapeutic regimens may be combined with the administration of the anti-HER2 antibody or a fragment thereof. The combined administration includes co-administration, using separate formulations or a single pharmaceutical formulation, and consecutive administration in either order, wherein preferably there is a time period while both (or all) active agents simultaneously exert their biological activities.

It may also be desirable to combine administration of the anti-HER2 antibody or a fragment thereof, with administration of an antibody or a fragment thereof directed against another tumor associated antigen. The other antibody in this case may, for example, bind to EGFR, ErbB3, ErbB4, or vascular endothelial growth factor (VEGF).

In one embodiment, the treatment of the present invention involves the combined administration of an anti-HER2 antibody or a fragment thereof and one or more chemotherapeutic agents or growth inhibitory agents, including co-administration of cocktails of different chemotherapeutic agents. Preferred chemotherapeutic agents include taxanes (such as paclitaxel and docetaxel) and/or anthracycline antibiotics. Preparation and dosing schedules for such chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992). The antibody may be combined with an anti-hormonal compound, e.g., an anti-oestrogen compound such as tamoxifen, an anti-progesterone such as onapristone (see EP616812), or an anti-androgen such as fiutamide, in dosages known for such molecules. Where the cancer to be treated is hormone independent cancer, the subject may previously have been subjected to anti-hormonal therapy and, after the cancer becomes hormone independent, the anti-HER2 antibody or a fragment thereof (and optionally other agents as described herein) may be administered to the subject.

In some cases it may be beneficial to also co-administer a cardioprotectant (to prevent or reduce myocardial dysfunction associated with the therapy) or one or more cytokines to the subject. One may also co-administer an EGFR-targeted drug or an anti-angiogenic agent. In addition to the above therapeutic regimes, the subject may be subjected to surgical removal of cancer cells and/or radiation therapy.

The present invention will now be illustrated, merely by way of example, with reference to the following methods and examples.

### EXAMPLES

### Example 1: Development of a Liquid Formulation

The purpose of this work was to determine the main formulation conditions that have an impact on the stability of the anti-HER2 antibody in a liquid formulation, which can be used as the basis for a lyophilized formulation of the antibody.

### General Materials & Methods

The anti-HER2 antibody was produced in wave bioreactors and captured by Protein A using MabSelect Sure (GE Healthcare; www.gelifesciences.com). The product was polished by preparative size exclusion chromatography using the column HiLoad 16/60 Sephadex 200 pg, 120 ml (GE Healthcare) with the running buffer 10 mM citrate pH 5.5. The product was concentrated to 50 mg/ml by UFDF before the addition of excipients. CEX-HPLC was performed with a column Bakerbond Wide-Pore CBX 5 μm 4.6×250 mm 300A (JT Baker 7114-00; www.jtbaker.nl) followed by SEC-HPLC using the column BioSep S3000 300 mm×4.6 mm, 300A, separation range: 5-500 kDa (Phenomenex OOH-2146-E0; www.phenomenex.com).

The following chemicals were used in the formulation screening: sodium acetate trihydrate (Merck; www.merckmillipore.com), acetic acid (Sigma; www.sigmaaldrich.com), sodium citrate (Sigma), ammonium sulphate (Fluka; www.sigmaaldrich.com), sodium chloride (Merck), hydrochloric acid 37% (Sigma) and sodium hydroxide 50% (Sigma). The following excipients were used in the formulation screening: Tween80™ (VWR; https://uk.vwr.com), sorbitol (Sigma), PEG8000 (Sigma), glycerol (VWR prolabo), sucrose (Sigma), mannitol (Merck) and glycine (Merck), and the amino acids: glutamine (Fluka), proline (Merck), leucine (Merck), aspartic acid (Merck), methionine (Sigma) and histidine (Sigma). All chemicals and excipients used during these screens were pharmacopeia grade (US or EP), except for sodium hydroxide 50% solution, and are suitable as parenterals. Buffer and excipients were filtered through a 0.22 μm filter before use.

1.1: Screen 1

Aim:

The conditions tested were: pH, buffer concentration, tonicifier concentration, surfactant concentration, amino acids and polyols.

Materials & Methods:

The anti-HER2 antibody was prepared as detailed above in the General Materials & Methods section. Formulated samples of the anti-HER2 antibody were incubated at 5±3° C. in a cold room, at 22±5° C. (laboratory room temperature, uncontrolled temperature) and at 37±1° C. in a cell culture incubator (Hera Cell 150, Thermo (R-022) or equivalent). The buffer exchange for the 50 mg/ml concentrated anti-HER2 antibody samples was performed with PD-10 columns (GE Healthcare). The formulation conditions were: pH 5.00 to 8.00, buffer concentration from 10 to 50 mM, amino acid concentration from 0 to 20 g/L, amino acid type (glutamine, proline, leucine, aspartic acid), sucrose 0 to 10 g/L, (NH₄)₂SO₄ concentration from 0 to 15 g/L and Tween 80™ concentration from 0 to 0.01%. The buffers tested were: acetate pH range 3.7-5.7; histidine pH range 5.0-7.0; citrate pH range 5.4-7.4 and phosphate pH range 6.2-8.2. The final volume of the anti-HER2 antibody was adjusted to reach the 21 mg/ml target concentration.

Procedure:

The anti-HER2 antibody was formulated at the target concentration of 21 g/L in 32 formulation formulations designed on the statistical software JMP® (SAS). The samples were incubated at 37±1° C. for 1 month and were analysed by SEC-HPLC and CEX-HPLC. A design of experiment approach was used with the statistical software JMP®. Table 1 overleaf shows the buffer, pH range and buffer concentrations tested.

Results:

Fragmentation and aggregation were visible in most samples; however the only parameter that had a major impact on the product stability was pH. In the pH range of pH 5.00 to 6.00, more preferably about pH 5.50, the maximal proportion of non-deamidated anti-HER2 antibody was obtained. This pH range (pH 5.00 to 6.00) was used in the further experiments detailed herein.

TABLE 1
JMP design for the first screening step. The design
produced 32 conditions with various concentrations
of buffer in a pH range of 5.00 to 8.00

| | | buffer conc | |
|---|---|---|---|
| No | pH | (mM) | buffer |
| 1 | 5.45 | 18.0 | acetate |
| 2 | 7.40 | 10.0 | phosphate |
| 3 | 5.00 | 10.0 | acetate |
| 4 | 8.00 | 30.0 | phosphate |
| 5 | 7.55 | 50.0 | phosphate |
| 6 | 8.00 | 10.0 | phosphate |
| 7 | 8.00 | 44.0 | phosphate |
| 8 | 7.70 | 40.0 | phosphate |
| 9 | 6.50 | 50.0 | histidine |
| 10 | 5.60 | 50.0 | histidine |
| 11 | 5.00 | 10.0 | acetate |
| 12 | 7.55 | 34.0 | phosphate |
| 13 | 5.45 | 14.0 | acetate |
| 14 | 8.00 | 18.0 | phosphate |
| 15 | 5.90 | 10.0 | histidine |
| 16 | 6.20 | 26.0 | histidine |
| 17 | 5.45 | 32.0 | acetate |
| 18 | 5.00 | 50.0 | acetate |
| 19 | 7.40 | 16.0 | phosphate |
| 20 | 6.80 | 26.0 | histidine |
| 21 | 5.00 | 16.0 | acetate |
| 22 | 7.40 | 50.0 | phosphate |
| 23 | 5.00 | 28.0 | acetate |
| 24 | 6.50 | 16.0 | phosphate |
| 25 | 7.25 | 34.0 | phosphate |
| 26 | 8.00 | 16.0 | phosphate |
| 27 | 5.00 | 44.0 | acetate |
| 28 | 5.45 | 44.0 | acetate |
| 29 | 6.35 | 44.0 | histidine |
| 30 | 5.60 | 42.0 | histidine |
| 31 | 8.00 | 50.0 | phosphate |
| 32 | 6.65 | 42.0 | histidine |

### 1.2: Screen 2

### Aim:

Various excipients were tested for their efficiency in stabilizing the product in the optimal pH range determined in Screen 1.

### Materials & Methods:

The anti-HER2 antibody was prepared as detailed above in the General Materials & Methods section. Formulated samples of the anti-HER2 antibody were incubated at 37±1° C. in a cell culture incubator, Hera Cell 150, Thermo (R-022) or equivalent. Buffer exchange for the 50 mg/ml concentrated anti-HER2 antibody samples was performed with PD-10 columns (GE Healthcare). The final volume was adjusted to reach the 21 mg/ml target concentration. The parameters evaluated were: various concentrations of asparagine, sucrose, PEG8000, ammonium sulphate, sodium chloride, glutamine, ethanol and Tween 80™.

### Procedure:

The anti-HER2 antibody was formulated at the target concentration of 21 g/L or 50 g/L. The tested formulations are shown in Table 2 overleaf. Various excipients were tested for their efficiency in stabilizing the product. The samples were stressed by an accelerated study at 37° C. for one month and analyzed by SEC-HPLC and CEX-HPLC.

### Results:

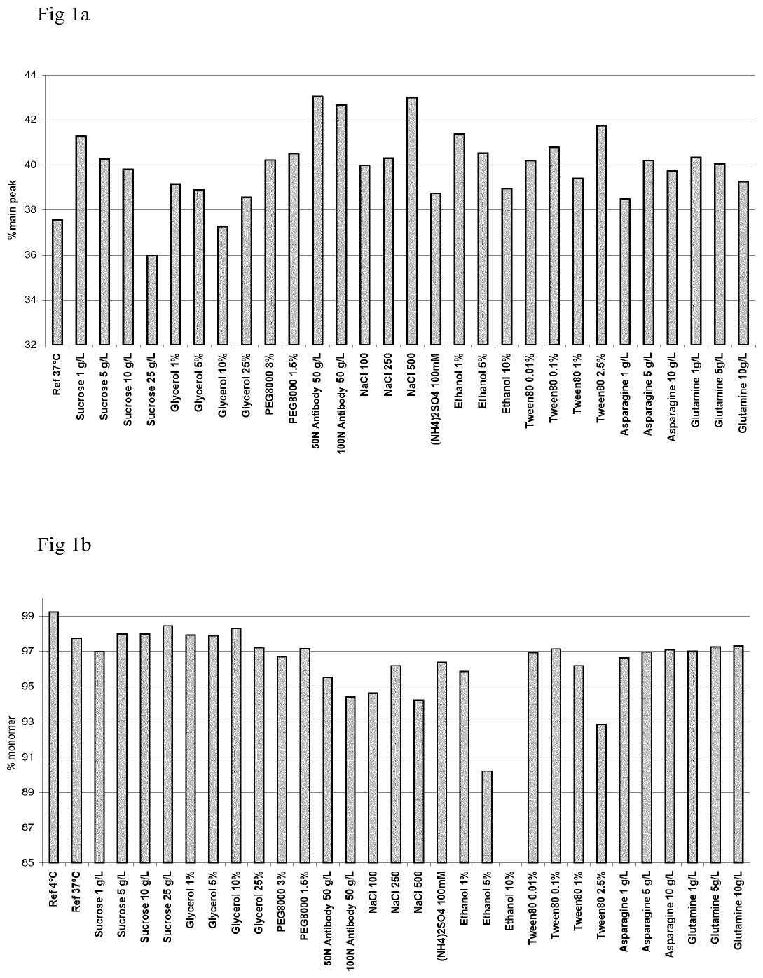

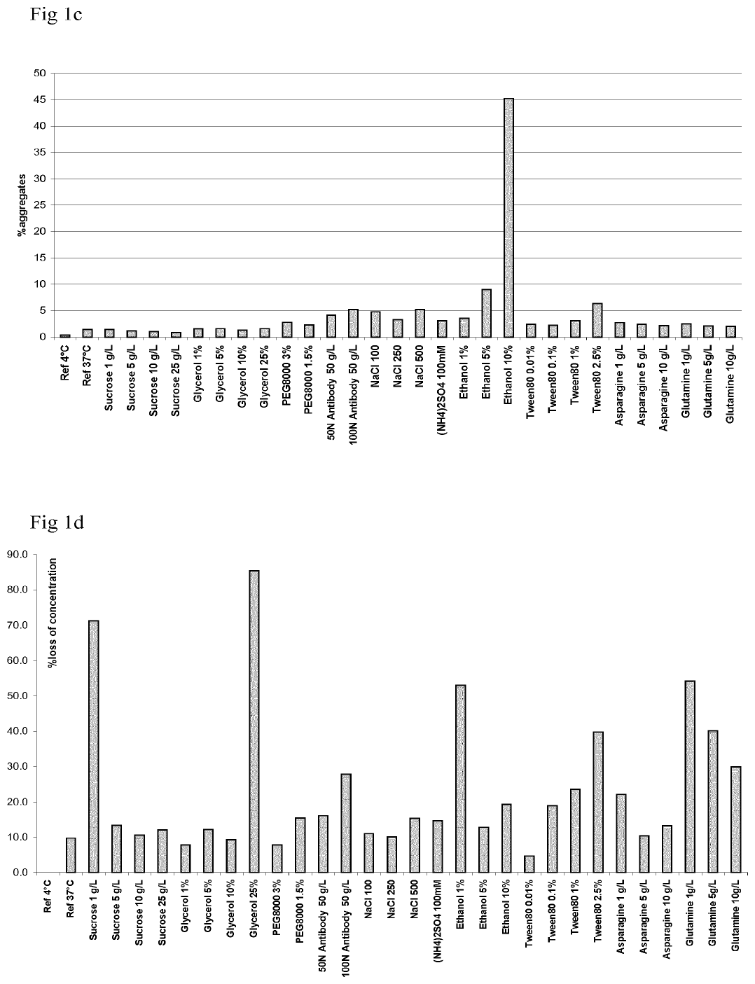

FIG. 1a shows that the variations of deamidation rate formulations were small. At time 0, the product had 75% of the non-deamidated form and after one month at 37° C., all the samples had from 36% to 43% of non-deamidated form. Formulation of the anti-HER2 antibody at a high concentration in the presence of a high concentration of Tween80™ or in a high salt concentration produced the best deamidation results. FIGS. 1b, c and d show that the conditions producing the lowest deamidation produced the highest aggregation levels and also the highest loss of antibody solubility. In conclusion, it was possible to reduce the deamidation rate to a low extent by using extreme formulation compositions. These included high concentrations of protein, salt and surfactant. The best conditions produced an increase of 6% of the non-deamidated form; however the same conditions produced the largest proportion of aggregation and an important loss in solubility.

TABLE 2
All samples were in 10 mM citrate pH 5.5 and 50
mM NaCl to which were added various excipients.
The antibody was formulation at 21 g/L or 50 g/L.

| Sample | Base | Formulation | Mab |
|---|---|---|---|
| No | composition | composition | concentration (g/L) |
| 1 | 10 mM citrate | Sucrose 1 g/L | 21 |
| 2 | pH 5.5 | Sucrose 5 g/L | 21 |
| 3 | 50 mM NaCl | Sucrose 10 g/L | 21 |
| 4 | (all compositions) | Sucrose 25 g/L | 21 |
| 5 | | Glycerol 1% | 21 |
| 6 | | Glycerol 5% | 21 |
| 7 | | Glycerol 10% | 21 |
| 8 | | Glycerol 25% | 21 |
| 9 | | PEG8000 3% | 21 |
| 10 | | PEG8000 1.5% | 21 |
| 11 | | NaCl 50 mM | 50 |
| 12 | | NaCl 100 mM | 50 |
| 13 | | NaCl 100 mM | 21 |
| 14 | | NaCl 250 mM | 21 |
| 15 | | NaCl 500 mM | 21 |
| 16 | | (NH₄)₂SO₄ 100 mM | 21 |
| 17 | | Ethanol 1% | 21 |
| 18 | | Ethanol 5% | 21 |
| 19 | | Ethanol 10% | 21 |
| 20 | | Tween80 0.01% | 21 |
| 21 | | Tween80 0.1% | 21 |
| 22 | | Tween80 1.0% | 21 |
| 23 | | Tween80 2.5% | 21 |
| 24 | | Asparagine 1 g/L | 21 |
| 25 | | Asparagine 5 g/L | 21 |
| 26 | | Asparagine 10 g/L | 21 |
| 27 | | Glutamine 1 g/L | 21 |
| 28 | | Glutamine 5 g/L | 21 |
| 29 | | Glutamine 10 g/L | 21 |

### 1.3: Screen 3

### Aim:

The aim of this screen was to reduce the deamidation of an anti-HER2 antibody by fine tuning the best parameters from Screen 2.

### Materials & Method:

The anti-HER2 antibody was prepared as detailed above in the General Materials & Methods section. For accelerated studies, the formulated anti-HER2 antibody samples were incubated at 37±1° C. in a cell culture incubator, Hera Cell 150, Thermo (R-022) or equivalent. The parameters tested were: pH 4.00 to pH 6.00, buffer types were acetate or citrate and the salt concentration was either 100 mM or 250 mM. The conditions are detailed in Table 3 overleaf. From a starting sample concentration of 84 mg/ml concentrated solutions of NaCl and buffer were added to reach the desired concentrations. The final volume was adjusted to reach the 21 mg/ml, 42 mg/ml or 63 mg/ml target concentrations of anti-HER2 antibody.

### Procedure:

The anti-HER2 antibody was formulated at the target concentrations of 21 mg/ml, 42 mg/ml or 63 mg/ml. Two buffer types with acidic pKas were evaluated for their efficiency in stabilizing the product in the optimal pH range of pH 4.00 to pH 6.00. The samples were stressed for one month in an accelerated study at 37° C. and analyzed by SEC-HPLC and CEX-HPLC.

### Results:

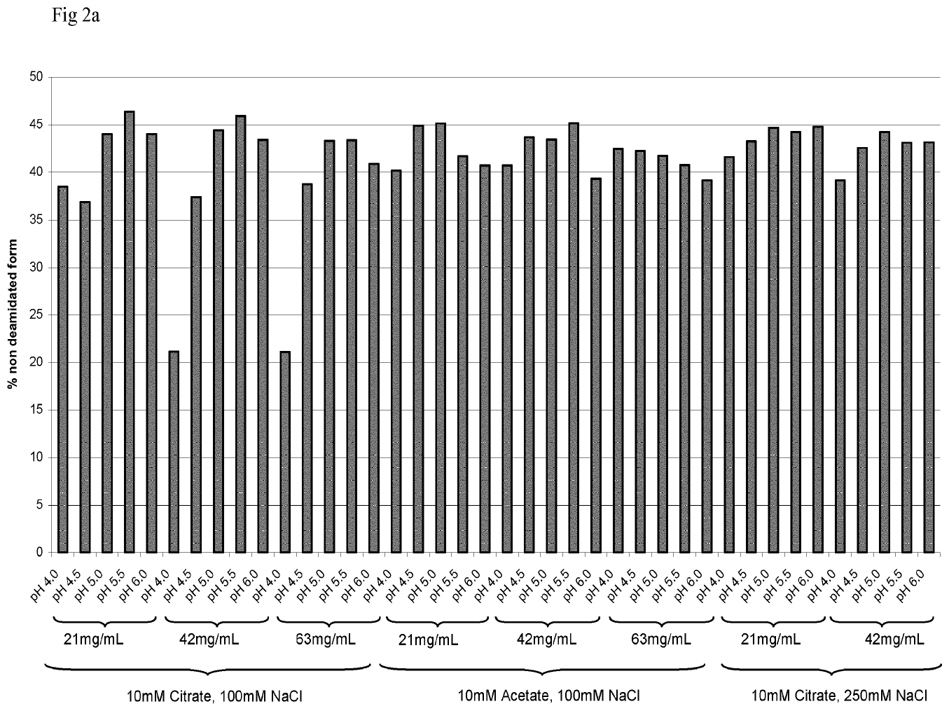

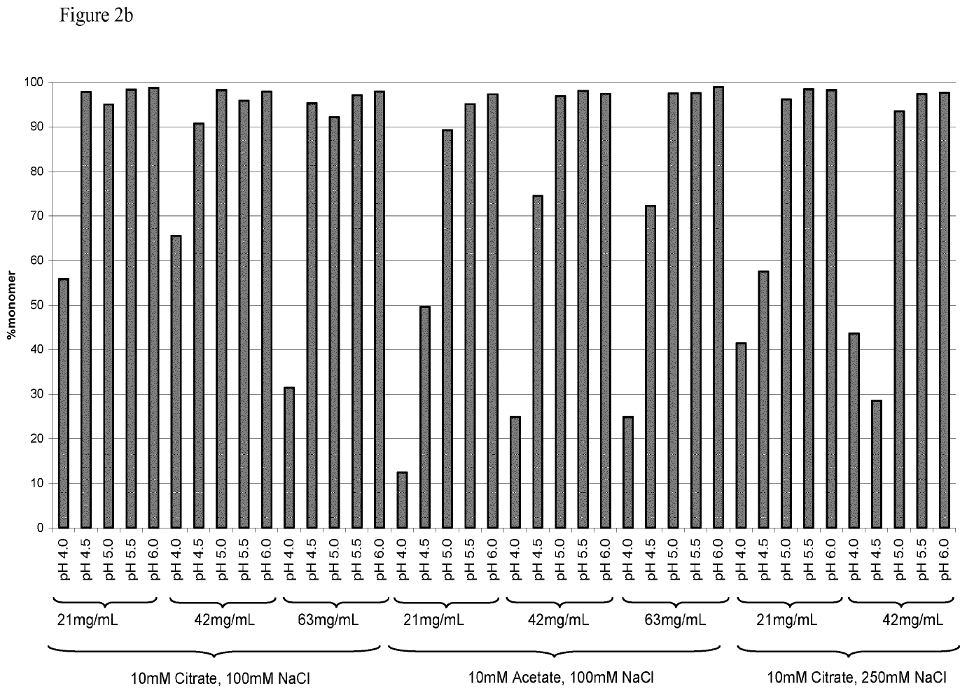

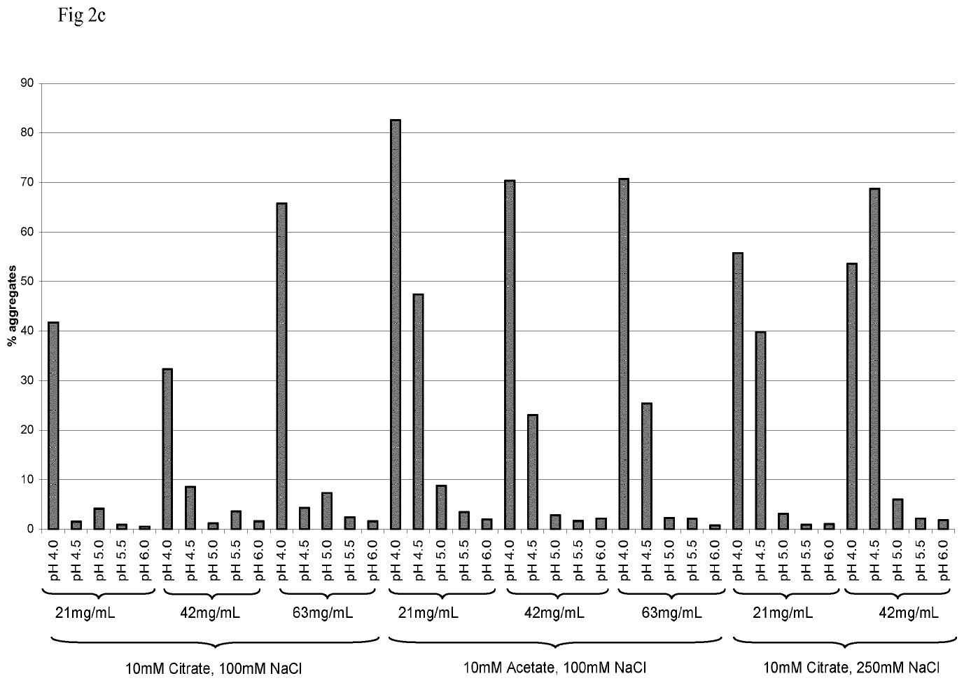

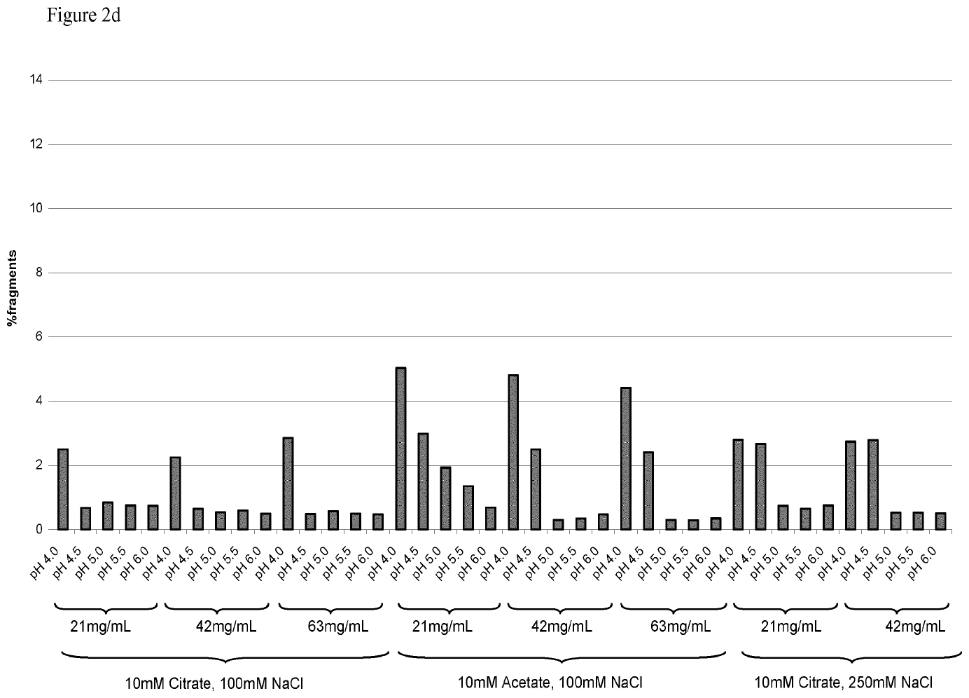

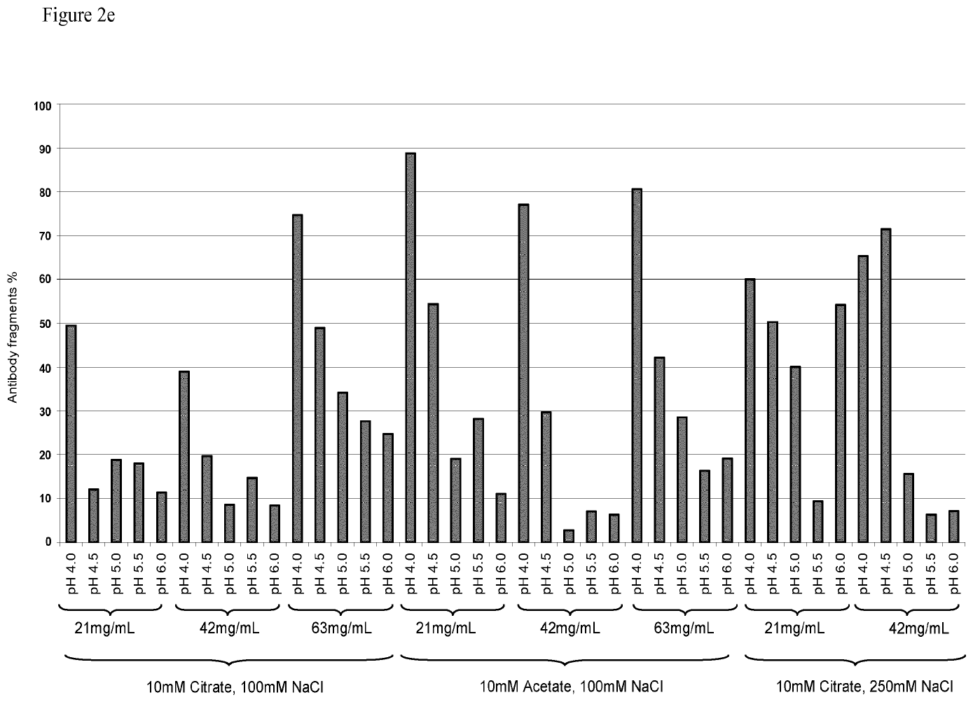

From the previous screens it was observed that the best anti-HER2 antibody stability results were obtained at mildly acidic pH values. The deamidation could be mostly inhibited in the presence of higher antibody concentration or salt concentration. In this screen, formulations of the anti-HER2 antibody at high antibody concentration or salt concentration and at a mildly acidic pH were evaluated. At time 0, the product had 75% of non-deamidated form. In FIG. 2a, the CEX-HPLC results shown that the maximal percentage of non-deamidated form reduced to 45%. The deamidation was more important at the very low pH values and an optimal pH was found to be in the range of pH 5.0 to pH6.0. The extent of deamidation at low pH was reduced when the buffer was acetate as opposed to citrate. NaCl at a high concentration (250 mM) reduced deamidation at low pH values. Altering the concentration of the anti-HER2 antibody in the formulations did not impact the results.

The SEC-HPLC results (FIGS. 2b, c, d & e) showed a maximum degradation at low pH values and high salt concentrations. At pH 4.0 and pH 4.5 there were minimal amounts of antibody monomeric form, high aggregation and high fragmentation. These low pH degradations were more pronounced when the buffer was acetate. The best results were obtained at pH 5.5 and pH 6.0, where acetate and citrate were found to be equivalent in stabilizing the anti-HER2 antibody. NaCl at 250 mM produced more degradation at low pH values than NaCl at 100 mM. The concentration of anti-HER2 antibody did not produce any difference.

TABLE 3
Testing conditions for Screen 3 to evaluate two buffers:
citrate and acetate in a pH range of pH 4.0 to pH 6.0

| | antibody | | | |
|---|---|---|---|---|
| No | conc. (mg/ml) | Buffer | pH | NaCl (mM) |
| 1 | 21 | 10 mM citrate | 4.0 | 100 |
| 2 | 21 | | 4.5 | 100 |
| 3 | 21 | | 5.0 | 100 |
| 4 | 21 | | 5.5 | 100 |
| 5 | 21 | | 6.0 | 100 |
| 6 | 42 | 10 mM citrate | 4.0 | 100 |
| 7 | 42 | | 4.5 | 100 |
| 8 | 42 | | 5.0 | 100 |
| 9 | 42 | | 5.5 | 100 |
| 10 | 42 | | 6.0 | 100 |
| 11 | 63 | 10 mM citrate | 4.0 | 100 |
| 12 | 63 | | 4.5 | 100 |
| 13 | 63 | | 5.0 | 100 |
| 14 | 63 | | 5.5 | 100 |
| 15 | 63 | | 6.0 | 100 |
| 16 | 21 | 10 mM acetate | 4.0 | 100 |
| 17 | 21 | | 4.5 | 100 |
| 18 | 21 | | 5.0 | 100 |
| 19 | 21 | | 5.5 | 100 |
| 20 | 21 | | 6.0 | 100 |
| 21 | 42 | 10 mM acetate | 4.0 | 100 |
| 22 | 42 | | 4.5 | 100 |
| 23 | 42 | | 5.0 | 100 |
| 24 | 42 | | 5.5 | 100 |
| 25 | 42 | | 6.0 | 100 |
| 26 | 63 | 10 mM acetate | 4.0 | 100 |
| 27 | 63 | | 4.5 | 100 |
| 28 | 63 | | 5.0 | 100 |
| 29 | 63 | | 5.5 | 100 |
| 30 | 63 | | 6.0 | 100 |
| 31 | 21 | 10 mM citrate | 4.0 | 250 |
| 32 | 21 | | 4.5 | 250 |
| 33 | 21 | | 5.0 | 250 |
| 34 | 21 | | 5.5 | 250 |
| 35 | 21 | | 6.0 | 250 |
| 36 | 42 | 10 mM citrate | 4.0 | 250 |
| 37 | 42 | | 4.5 | 250 |
| 38 | 42 | | 5.0 | 250 |
| 39 | 42 | | 5.5 | 250 |
| 40 | 42 | | 6.0 | 250 |

In conclusion, the most important parameter impacting the formulation was the pH of the sample. The best stability pH range was found in the range of pH 5.5 and pH 6.0. The antibody concentration did not produce a significant difference on deamidation.

### 1.4: Screen 4

### Aim:

The aim of this step was to define a final liquid formulation that could be used as the basis for the lyophilized formulation. The first step consisted of narrowing down the best parameters to their optimal range. The second step was the design of an experiment using statistical software for selecting a single final composition for the anti-HER2 antibody liquid formulation.

### Materials & Methods:

Anti-HER2 antibody was prepared as detailed above in the General Materials & Methods section. For accelerated studies, the formulated samples of the anti-HER2 antibody were incubated at 5±3° C. in a cold room, at 22±5° C. (laboratory room temperature, uncontrolled temperature) and 37±1° C. in a cell culture incubator, Hera Cell 150, Thermo (R-022) or equivalent. The statistical software used was JMP® (SAS).

### Procedure:

Preliminary screenings were performed in order to narrow down the parameters for the experimental design. The buffer screening was performed by dialysing the anti-HER2 antibody in NaCl 50 mM with the addition of acetate or citrate at pH 5.5 at the following concentrations: 1.0, 2.5, 5.0, 7.5, 10.0, 15.0 and 20.0 mM. The optimal salt concentration was determined by dialysing the anti-HER2 antibody in 10 mM citrate pH 5.5 with the addition of the following concentrations of NaCl: 0, 10, 50, 100, 200, 400, 600, 800 and 1000 mM. The antioxidant methionine was tested at the concentrations of 1, 4 or 8 mM in the anti-HER2 antibody dialysed in 5 mM Citrate pH 5.5, 50 mM NaCl. The experiment was performed according to a statistical design. The parameter ranges used in the statistical software JMP® were: pH 5.50 to pH 6.25, buffer concentration from 0 to 20 mM, NaCl concentration from 50 mM to 250 mM, a polyol (either sorbitol, glycerol or none), an amino acid (proline, glycine or none), Tween80™ 0% or 0.01%, methionine at 0 mM or 4 mM. The design of the experimental approach is shown in Table 4 below.

TABLE 4
JMP ® statistical software design for the experiment. The design produced 24
conditions.

| | | Acetate | | Sugar | Amino acid | | Methionine |
|---|---|---|---|---|---|---|---|
| No | NaCl (mM) | (mM) | pH | (5%) | (20 g/L) | Tween80 ® % | (mM) |
| 1 | 50 | 15.3 | 6.0 | Sorbitol | none | 0.010 | 0 |
| 2 | 230 | 7.7 | 6.1 | Sorbitol | Pro | 0.010 | 4 |
| 3 | 50 | 1.0 | 6.2 | Glycerol | none | 0.000 | 0 |
| 4 | 180 | 7.7 | 5.9 | Glycerol | Gly | 0.000 | 0 |
| 5 | 120 | 7.7 | 5.7 | Glycerol | Gly | 0.010 | 4 |
| 6 | 250 | 11.5 | 5.6 | none | Gly | 0.010 | 4 |
| 7 | 120 | 13.4 | 5.6 | Sorbitol | none | 0.000 | 4 |
| 8 | 180 | 1.0 | 5.9 | Sorbitol | Pro | 0.000 | 4 |
| 9 | 150 | 2.9 | 5.5 | none | none | 0.000 | 0 |
| 10 | 250 | 20.0 | 6.2 | Sorbitol | Gly | 0.000 | 4 |
| 11 | 220 | 20.0 | 6.0 | none | Pro | 0.010 | 0 |
| 12 | 50 | 5.8 | 5.4 | Glycerol | Pro | 0.010 | 4 |
| 13 | 80 | 20.0 | 5.7 | none | none | 0.010 | 4 |
| 14 | 250 | 2.9 | 5.7 | none | none | 0.000 | 0 |
| 15 | 230 | 15.3 | 5.6 | Glycerol | Gly | 0.000 | 0 |
| 16 | 190 | 13.4 | 5.6 | Sorbitol | Pro | 0.010 | 0 |
| 17 | 70 | 12.4 | 5.5 | Sorbitol | Gly | 0.000 | 4 |
| 18 | 70 | 18.1 | 6.2 | none | Pro | 0.000 | 0 |
| 19 | 140 | 18.1 | 5.9 | none | Gly | 0.010 | 4 |
| 20 | 250 | 18.1 | 6.2 | Sorbitol | Gly | 0.010 | 0 |
| 21 | 50 | 20.0 | 6.0 | Glycerol | Pro | 0.000 | 4 |
| 22 | 240 | 1.0 | 6.2 | Glycerol | none | 0.010 | 4 |
| 23 | 80 | 3.9 | 6.1 | none | Gly | 0.000 | 4 |
| 24 | 70 | 17.2 | 5.5 | Sorbitol | Gly | 0.010 | 0 |

### Results:

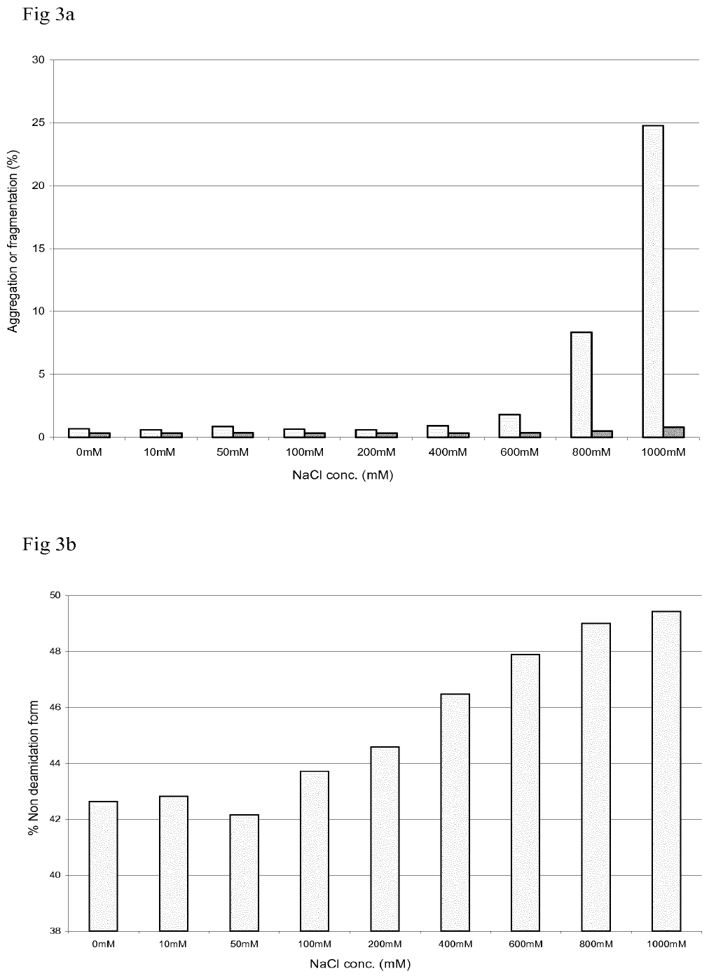

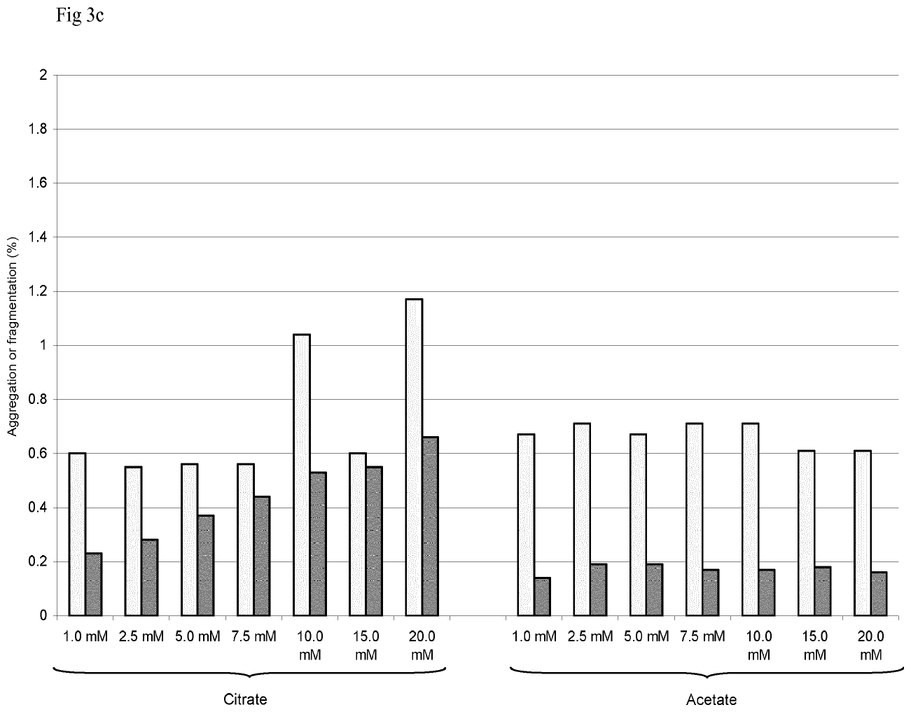

The results are shown in FIG. 3 and Table 5. FIG. 3a shows that the aggregation and fragmentation increased at NaCl concentrations higher than 200 mM. FIG. 3b shows that the proportion of non deamidated forms was lowest at 0 mM to 50 mM NaCl and then increased linearly with the salt concentration. FIG. 3c showed that at pH 5.5, an acetate buffer resulted in less fragments and aggregates. The antioxidant methionine had no impact on the stability of the anti-HER2 antibody.

TABLE 5
Anti-HER2 antibody formulated with an antioxidant: percentage
of monomers, aggregation, fragmentation and non deamidated form

| Methionine | Conc | % | % | % | % non- |
|---|---|---|---|---|---|
| (mM) | (mg/ml) | monomer | dimer | fragment | deamidated |
| 0.0 | 17.828 | 98.56 | 0.86 | 0.37 | 42.16 |
| 1.0 | 18.230 | 98.83 | 0.61 | 0.36 | 42.03 |
| 4.0 | 18.473 | 98.74 | 0.87 | 0.39 | 41.50 |
| 8.0 | 18.374 | 98.56 | 0.81 | 0.39 | 41.24 |

Therefore, for the final experimental design, it was decided to include NaCl in a range of 50 mM to 250 mM and an acetate buffer in a range of 1.0 mM to 20.0 mM. A single concentration of methionine was also included at a concentration of 4.0 mM.

The final liquid formulation composition of the anti-HER2 antibody was determined in an experiment using the statistical software JMP® as shown in Table 4. The samples were incubated at 37° C. for 1 month and were analyzed by SEC-HPLC and CEX-HPLC. The results were entered in the statistical software, which detected statistical significance (P<0.05) for the percentage of monomers, percentage of aggregates and product concentration (results not shown). The responses were of excellent quality with R squares of 1.00, small probability of wrong rejection of the null hypothesis (P<0.05) and small RMSE values. The fragments response had a R square of 0.94 but the RMSE and P values were large. The percentage of fragments had no statistically significant relation to any buffer composition.

The prevision of profiles (results not shown) indicated that the polyol, amino acid, methionine and Tween80™ had very large error bars and no conclusions could be made. pH, buffer concentration and salt concentration had the largest impact on the product stability. The pH was optimal for minimum aggregation in the range of pH 5.9 to pH 6.1 but the optimum for minimum deamidation was at pH 5.5 to pH 5.8. The optimal NaCl concentration for low aggregation was in the range of 75 mM to 175 mM and the optimum for minimum deamidation was 200 mM to 225 mM. The optimal buffer concentrations predicted for minimal aggregation were in the range of 7.5 mM to 17.5 mM and the optimum for minimal deamidation were with buffer concentrations less than 5 mM. In short, the optimum conditions for avoiding deamidation were different to the optimum conditions for avoiding aggregation.

During the screening experiments the aggregation levels were much lower than the deamidation levels. After a one month accelerated study at 37° C. at pH 5.5 the aggregation in the screenings discussed above was generally in the range of 0.5% while the deamidated form increased by 30%. It was decided to formulate in conditions that would reduce the largest degradation/deamidation. Therefore, it was decided that the final composition would contain: 4 mM acetate pH 5.7 and 214 mM NaCl. Since these conditions are less favourable for anti-HER2 antibody solubility, an excipient with solubilising properties was also included and proline at 20 g/L was added to the final composition. Finally, although not demonstrated, the presence of a surfactant is known to be desirable in a formulation to avoid product loss due to the mechanical stress encountered during transport (Mahler H-C et al. (2009) J Pharm Sci, 98(12): 4525-33). During the screening experiments Tween80™ at a low concentration improved the solubility and did not impact negatively the integrity of the product. Therefore Tween80™ at 0.01% was added to the final composition.

The parameter that was the most import for stability of the anti-HER2 antibody was pH and therefore an acetate buffer was chosen for the final concentration as this had been shown to confer greater stability on the formulation than a citrate buffer (see FIG. 3c). The optimal concentrations of tonicifier and buffer were detected in the optimal pH range. Amino acids were also tested with some having useful solubilising properties on the anti-HER2 antibody. The surfactants tested had no negative impact on the anti-HER2 antibody stability at lower concentrations and were therefore included in the final composition for protection against mechanical stress. The final composition is shown in Table 6.

TABLE 6
Anti-HER2 antibody liquid formulation
Liquid formulation

| | |
|---|---|
| anti-HER2 Ab | 21 g/L |
| pH | pH 5.7 |
| buffer | 4 mM acetate |
| tonicifier | 214 mM NaCl |
| surfactant | 0.01% Tween80 ™ |
| solubilizer | 20 g/L proline |

### Example 2: Development of a Lyophilized Formulation

The liquid formulation of the anti-HER2 antibody as determined in Example 1, was used at the starting point for developing an optimal lyophilized formulation of the same antibody. It was clearly demonstrated in the screens of Example 1, that the most important parameter for the stability of this anti-HER2 antibody in formulation was pH. Use of an acetate buffer in the form of sodium acetate was found to give optimal results compared to a citrate buffer and therefore the acetate buffer was included in the final liquid formulation. To further stabilise the formulation of the anti-HER2 antibody for lyophilisation a lyoprotectant was required. As detailed herein, many types of lyoprotectants are available and for the purposes of this experiment, the following lyoprotectants were initially selected: sucrose, mannitol, glucose, sorbitol and lactose. However, it is widely known that glucose, lactose and sorbitol are reducing sugars and therefore these lyoprotectants were not selected for further experimental work. For the first runs, mannitol was chosen as the lyoprotectant for the anti-HER2 antibody lyophilized formulation.

### General Materials & Methods

The following chemicals were used in the formulation screening: sodium acetate trihydrate (Merck), acetic acid (Sigma), hydrochloric acid 37% (Sigma) and sodium hydroxide 50% (Sigma). The following excipients were used in the formulation screening: Tween80™ (VWR), sucrose (Sigma), mannitol (Merck) and glycine (Merck). Stock solutions of sucrose (600 g/1), glycine (130 g/1), mannitol (150 g/1) and Tween80™ (10%) were prepared in 5.75 mM sodium acetate pH 5.7. HCl 37% and NaOH were used for pH adjustment. The excipients were then added to the anti-HER2 antibody formulation by dilution of the stock solution in the formulation to achieve the desired concentration. Specifications for the buffer were 5.7±0.1 for the pH and 0.42 to 0.66 mS/cm for the conductivity. ISO3696 Type II water was used throughout. This highly purified water was prepared in-house using an osmotic membrane and an ozone sterilization system (Christ Aqua, Switzerland). All chemicals and excipients were suitable for parenteral administration. The buffer was filtered through a 0.22 μm filter before use.

The anti-HER2 antibody test material used for this study was first purified using Protein A affinity chromatography using MabSelect Sure (GE Healthcare). As the purity of the product post protein A was high (monomer content >99%), no further purification steps were performed prior to diafiltration and concentration of the product.

Purified anti-HER2 antibody was diafiltrated 7-10 times by tangential flow filtration (TFF) in 5.75 mM sodium acetate pH 5.7 and then concentrated to 50-60 g/1. After TFF, the anti-HER2 antibody was formulated by the addition of sucrose, glycine and Tween80™ to reach the final buffer composition of 30 g/1. The reference standard material used for the analytical testing was DRS-anti-HER2 antibody and was kept at Quality Control at a temperature below −60° C.

To prepare the lyophilized drug product, a Telstar lyophilization system was used (Lyobeta 15 lyophilizer; Telstar, Terrassa, Spain). A VP600 Vötsch stability chamber (Vötsch Industrietechnik GmbH, Germany) was used for the 3 months study at 40±2° C., 50±5% relative humidity test and also for the 12 months study at 25±2° C., ±5% relative humidity test. A standard laboratory fridge was used for the 36 months stability at 5±3° C.

Vials and stopper materials used were those intended for clinical trial manufacture. Vials were 20 ml Fiolax clear, USP type I glass vials purchased from Schott (Art. No: 1156521) and the stoppers were Flurotec distributed by West Pharma (ref FD20TT3WRS).

Stability of the drug product was determined using a number of tests to evaluate changes in the drug product over time. CEX-HPLC (Dionax, ProPac WCX-104×250 mm), SEC-HPLC (Phenomenex, YARRA 3u SEC-3000 7.8×300 mm) and SDS-PAGE are sensitive indicators of product breakdown. The profile of drug product using these assays will change over time if the sample is subject to degradation. The A280 assay can be used to detect changes in the protein level which may be indicative of product breakdown or adhesion to the container wall.

An ELISA cell based assay and an ELISA binding assay can be used to determine the potency of the product, with a change in the potency of the sample being indicative of product deterioration. The residual moisture of the product was assessed by Karl Fisher assay.

### 2.1: Screen 1

### Aim:

To determine the effect of the lyoprotectant mannitol on antibody stability

### Materials & Method:

The formulations were prepared according to the General Materials & Methods section above. Two formulations were tested in this screen that contained mannitol with different concentrations of Tween80™. The excipients, buffer and antibody were more concentrated than in the liquid formulation (×1.43) to achieve 150 mg lyophilized product per vial and no more than 1 cm cake height. The formulations tested were:

30 mg/ml anti-HER2 antibody, 5.76 mM sodium acetate pH 5.7, 72 mg/ml mannitol, 0.014% Tween80™; and 30 mg/ml anti-HER2 antibody, 5.76 mM sodium acetate pH 5.7, 72 mg/ml mannitol, 0.043% Tween80™.

The lyophilization cycle performed is detailed in Table 7 below and also contained an annealing step to increase crystallinity.

TABLE 7
Lyophilization cycle parameters

| Step | Temp (° C.) | Vacuum (mbar) | Time (h) |
|---|---|---|---|
| Freezing | 5 | | 0.15 |
| Freezing | 5 | | 0.30 |
| Freezing | −20 | | 0.25 |
| Freezing | −20 | | 0.3 |
| Freezing | −45 | | 0.35 |
| Freezing | −45 | | 2.00 |
| Freezing | −10 | | 1.15 |
| Freezing | −10 | | 2.00 |
| Freezing | −45 | | 1.15 |
| Freezing | −45 | | 2.00 |
| Vacuum | | 0.1 | |
| Primary drying | −30 | 0.1 | 1.00 |
| Primary drying | −30 | 0.1 | 72.00 |
| Secondary drying | 25 | | 5.00 |
| Secondary drying | 25 | | 24.00 |

### Results:

The starting product contained 99.8% monomers after purification. After the first test runs, the product contained 98.0% monomers (and 2.0% dimers) and no difference in the percentage of monomers or dimers was observed with either formulation. Therefore the different concentrations of Tween80™ did not affect the percentage of monomers in the lyophilized formulation. In conclusion, the first run shows that mannitol had a low cryoprotective effect (decrease of monomer content). A free-thaw study will therefore be performed before the next lyophilization runs to select excipients based on their cryoprotective capabilities. Given the minimal effect of Tween concentration (which is supposed to prevent the formation of aggregates during the reconstitution of the cake), it was decided to continue using the lowest Tween80™ concentration (0.014%), in this study.

### 2.2: Screen 2

### Aim:

In order to determine the optimal conditions between cake appearance and stability, different mixtures of mannitol and sucrose were tested over five freeze-thaw cycles.

### Materials & Method:

The formulations were prepared according to the General Materials & Methods section above. The formulations tested are shown in Table 9 below and were subjected to five freeze-thaw cycles at −40° C. and 25° C. The formulations with the highest percentages of monomers were then selected for lyophilization using the lyophilization cycle as detailed in Table 7. Following lyophilization, the cakes were reconstituted and the percentage of monomers determined for each formulation.

### Results:

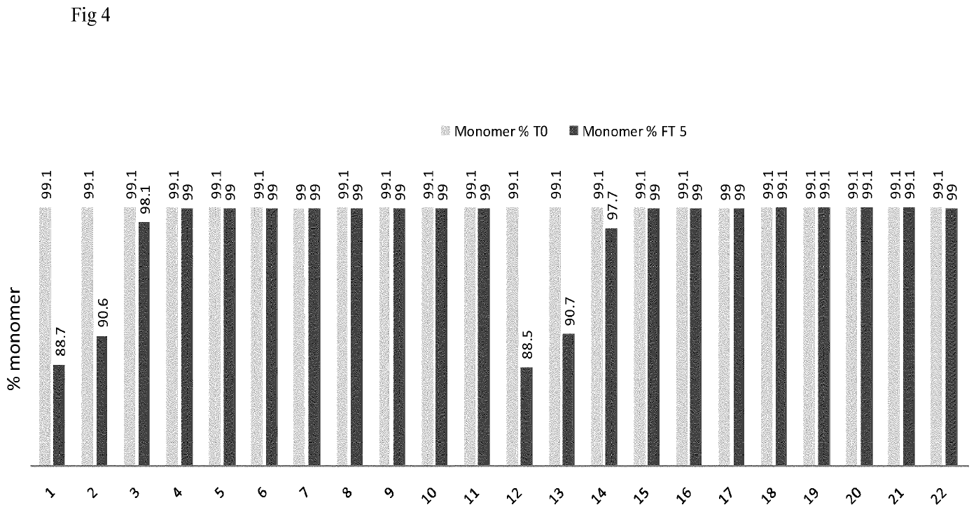

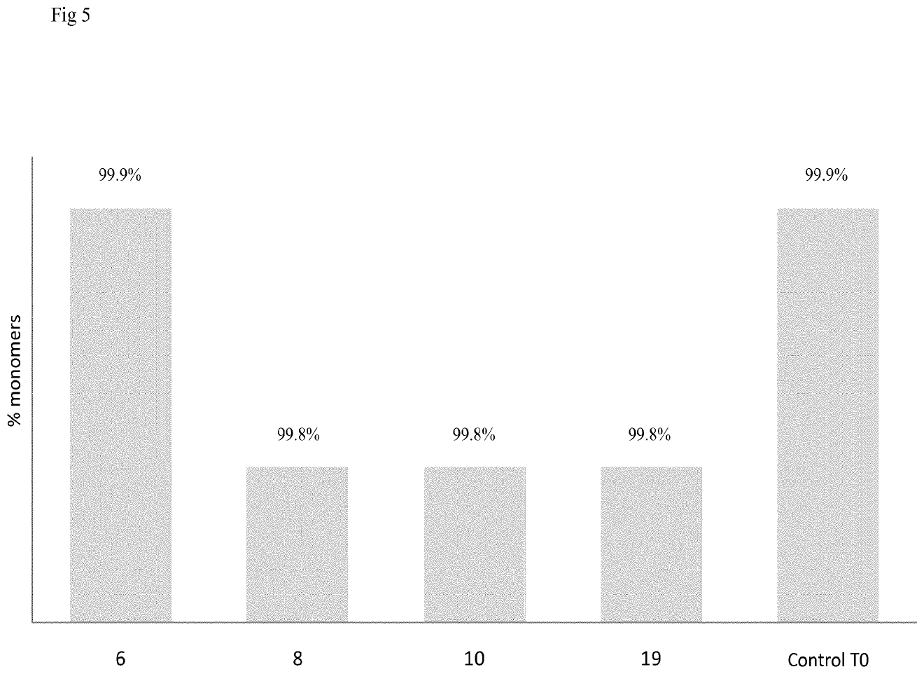

The results of five freeze-thaw cycles are shown in FIG. 4. Formulations 6, 8, 10 and 19 were selected for lyophilisation since these formulations had the highest percentages of monomers present after freeze-thawing. After lyophilisation, formulation 10 showed the best cake appearance (results not shown). After reconstitution of the cakes, the percentage of monomers was determined and the results are shown in FIG. 5. In conclusion, a combination of the lyoprotectants mannitol and sucrose imparted good stability on the lyophilized formulation of the anti-HER2 antibody; however the lyophilized formulations had a poor cake aspect. In order to test for improved formulation stability over these results, it was decided to test a higher concentration of sucrose. Furthermore, to try and improve the cake aspect, it was decided to test the addition of the bulking agent glycine to the formulation.

TABLE 8
Mannitol/sucrose formulations subjected to five freeze-thaw cycles

| No | Buffer | Mannitol % | Sucrose % | Tween80 ™ % |
|---|---|---|---|---|
| 1 | 5.76 mM | 6 | 0 | 0.014 |
| 2 | sodium | 4 | 0 | 0.014 |
| 3 | acetate | 2 | 0 | 0.014 |
| 4 | pH 5.7 | 0 | 4 | 0.014 |
| 5 | | 0 | 6 | 0.014 |
| 6 | | 1 | 6 | 0.014 |
| 7 | | 1 | 4 | 0.014 |
| 8 | | 2 | 6 | 0.014 |
| 9 | | 2 | 4 | 0.014 |
| 10 | | 4 | 6 | 0.014 |
| 11 | | 4 | 4 | 0.014 |
| 12 | | 6 | 0 | 0.043 |
| 13 | | 4 | 0 | 0.043 |
| 14 | | 2 | 0 | 0.043 |
| 15 | | 0 | 4 | 0.043 |
| 16 | | 0 | 6 | 0.043 |
| 17 | | 1 | 6 | 0.043 |
| 18 | | 1 | 4 | 0.043 |
| 19 | | 2 | 6 | 0.043 |
| 20 | | 2 | 4 | 0.043 |
| 21 | | 4 | 6 | 0.043 |
| 22 | | 4 | 4 | 0.043 |

2.3: Screen 3

### Aim:

To determine the moisture and cake appearance of the preferred formulation comprising 4% mannitol and 6% sucrose from Screen 2 and also to test higher concentrations of both lyoprotectants to establish if cake aspect could be improved. In addition, the effect of a mixture of mannitol and glycine on the stability of the formulations was tested.

### Materials & Method:

The formulations were prepared according to the General Materials & Methods section above. The formulations tested are shown in Table 9 below and were subjected to lyophilization according to the parameters in Table 7.

TABLE 9
Mannitol/sucrose/glycine formulations subjected to lyophilization

| No | Buffer | Mannitol % | Sucrose % | Glycine mM | Tween80 ™ % |
|---|---|---|---|---|---|
| 1 | 5.76 mM | 4 | 6 | 0 | 0.014 |
| 2 | sodium | 6 | 6 | 0 | 0.014 |
| 3 | acetate | 0 | 6 | 0 | 0.014 |
| 4 | pH 5.7 | 0 | 8 | 0 | 0.014 |
| 5 | | 0 | 10 | 0 | 0.014 |
| 6 | | 0 | 12 | 0 | 0.014 |
| 7 | | 5.6 | 0 | 3 | 0.014 |

### Results:

The percentage of moisture and cake appearance are described in Table 10 below for each formulation tested. Formulations 1, 2 and 7 showed good cake appearance; however formulation 2 had the highest moisture content. Formulations 3-6, which contained only sucrose had a shrunk and cracked cake appearance, although this was reduced at the higher sucrose concentrations of 10 and 12% (data not shown).

TABLE 10
Moisture content and cake appearance
of the seven formulations tested

| No | Moisture % | Cake Appearance |
|---|---|---|
| 1 | 0.49 | Good, no shrinkage, no cracking |
| 2 | 0.80 | Good, no shrinkage, no cracking |
| 3 | 0.60 | Shrinkage and cracking |
| 4 | 0.59 | Shrinkage and cracking |
| 5 | 0.45 | Shrinkage and cracking |
| 6 | 0.55 | Shrinkage and cracking |
| 7 | 0.38 | Good, no shrinkage, no cracking |

### 2.4: Screen 4

### Aim:

The aim of this screen was to determine the stability of a formulation of the anti-HER2 antibody containing mannitol, glycine and Tween80™ as excipients. The formulations would first be subjected to five cycles of freeze-thaw and the most stable formulations selected for further lyophilization.

### Materials & Method:

The formulations were prepared according to the General Materials & Methods section above. The formulations tested are shown in Table 11 below and were subjected to five freeze-thaw cycles at −40° C. and 25° C.

TABLE 11
Mannitol/glycine formulations subjected
to five cycles of freeze-thaw

| No | Buffer | Mannitol % | Glycine % | Tween80 ™ % |
|---|---|---|---|---|
| 1 | 5.76 mM | 2 | 0.022 | 0.014 |
| 2 | sodium | 4 | 0.022 | 0.014 |
| 3 | acetate | 6 | 0.022 | 0.014 |
| 4 | pH 5.7 | 2 | 0.400 | 0.014 |
| 5 | | 4 | 0.400 | 0.014 |
| 6 | | 6 | 0.400 | 0.014 |
| 7 | | 2 | 0.600 | 0.014 |
| 8 | | 4 | 0.600 | 0.014 |
| 9 | | 6 | 0.600 | 0.014 |
| 10 | | 2 | 1.000 | 0.014 |
| 11 | | 4 | 1.000 | 0.014 |
| 12 | | 6 | 1.000 | 0.043 |

### Results:

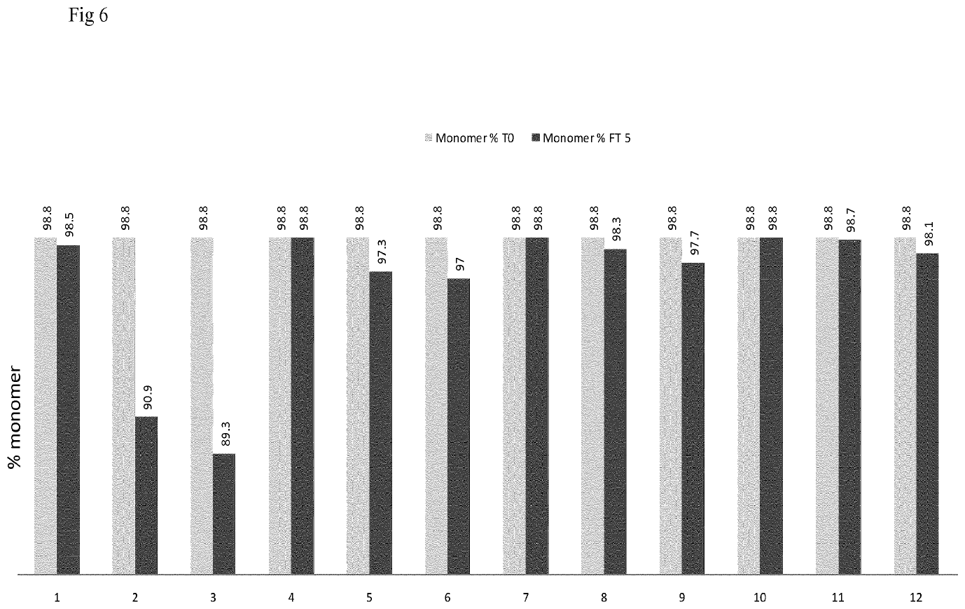

The stability of the 12 formulations of the anti-HER2 antibody tested in five freeze-thaw cycles is shown in FIG. 6, as the percentage of monomer present. It was observed during this study, that the ratio of mannitol:glycine appeared to be important for the stabilisation of the anti-HER2 antibody. Formulations 1, 4, 7, 10 and 11 were selected for further lyophilisation since the ratios of mannitol:glycine used had little effect on stability of the formulation before and after five cycles of freeze-thaw.

### 2.5: Screen 5

### Aim:

Since initial good stability results were observed with the mannitol/glycine formulations subjected to freeze-thaw cycles in Screen 4, it was decided to test a mixture of sucrose/glycine formulations and high sucrose concentration formulations alongside the selected mannitol/glycine formulations in a lyophilization cycle.

### Materials & Method:

The formulations were prepared according to the General Materials & Methods section above. The formulations tested are shown in Table 12 below and were subjected to lyophilisation according to the parameters in Table 7.

TABLE 12
Concentrations of the excipients tested
in a lyophilization cycle in Screen 5

| No | Buffer | Mannitol % | Sucrose % | Glycine % | Tween80 ™ % |
|---|---|---|---|---|---|
| 1 | 5.76 mM | 0 | 10 | 0 | 0.014 |
| 2 | sodium | 0 | 12 | 0 | 0.014 |
| 3 | acetate | 0 | 10 | 0.4 | 0.014 |
| 4 | pH 5.7 | 0 | 10 | 0.6 | 0.014 |
| 5 | | 0 | 10 | 1.0 | 0.014 |
| 6 | | 0 | 12 | 0.4 | 0.014 |
| 7 | | 0 | 12 | 0.6 | 0.014 |
| 8 | | 0 | 12 | 1.0 | 0.014 |
| 9 | | 1 | 10 | 0 | 0.014 |
| 10 | | 2 | 10 | 0 | 0.014 |
| 11 | | 4 | 10 | 0 | 0.014 |
| 12 | | 1 | 12 | 0 | 0.014 |
| 13 | | 2 | 12 | 0 | 0.014 |
| 14 | | 4 | 12 | 0 | 0.014 |
| 15 | | 2 | 0 | 0.4 | 0.014 |
| 16 | | 2 | 0 | 0.6 | 0.014 |
| 17 | | 2 | 0 | 1.0 | 0.014 |
| 18 | | 4 | 0 | 1.0 | 0.014 |

### Results:

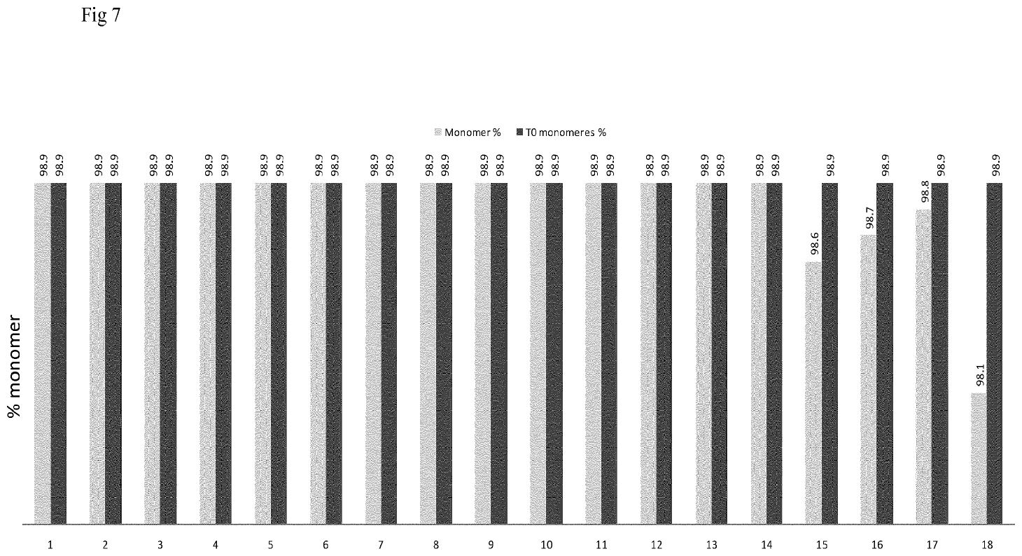

The stability of the 12 formulations of anti-HER2 antibody tested in five cycles of freeze-thaw is shown in FIG. 7 as the percentage of monomer present. Cake appearance was also assessed (results not shown) and the seven formulations 4, 8, 10, 12, 15-17, which showed best stability and cake appearance, were selected for a one month stability study at 40° C.

### 2.5: Screen 6

### Aim:

To determine the stability of seven lyophilized anti-HER2 antibody formulations at 40° C. for 1 month.

### Materials & Method:

The formulations were prepared according to the General Materials & Methods section above. Seven formulations containing the excipients as listed in Table 13 were subjected to a lyophilization cycle according to the parameters as set out in Table 14.

TABLE 13
Composition of the formulations subjected to lyophilization cycle

| No | Buffer | Mannitol % | Sucrose % | Glycine % | Tween80 ™ % |
|---|---|---|---|---|---|
| 1 | 5.76 mM | 0 | 10 | 0.6 | 0.014 |
| 2 | sodium | 0 | 12 | 1.0 | 0.014 |
| 3 | acetate | 2 | 10 | 0.0 | 0.014 |
| 4 | pH 5.7 | 1 | 12 | 0.0 | 0.014 |
| 5 | | 2 | 0 | 0.4 | 0.014 |
| 6 | | 2 | 0 | 0.6 | 0.014 |
| 7 | | 2 | 0 | 1.0 | 0.014 |

TABLE 14
Lyophilization cycle parameters

| Step | Temp (° C.) | Vacuum (mbar) | Time (h) |
|---|---|---|---|
| Freezing | 5 | | 0.20 |
| Freezing | 5 | | 0.30 |
| Freezing | 0 | | 0.05 |
| Freezing | 0 | | 0.30 |
| Freezing | −45 | | 0.45 |
| Freezing | −45 | | 2.00 |
| Freezing/annealing | −15 | | 0.30 |
| Freezing/annealing | −15 | | 2.00 |
| Freezing | −45 | | 0.30 |
| Freezing | −45 | | 2.00 |
| Vacuum | | 0.1 | |
| Primary drying | −30 | 0.1 | 1.00 |
| Primary drying | −30 | 0.1 | 72.00 |
| Secondary drying | 25 | | 5.00 |
| Secondary drying | 25 | | 24.00 |

### Results:

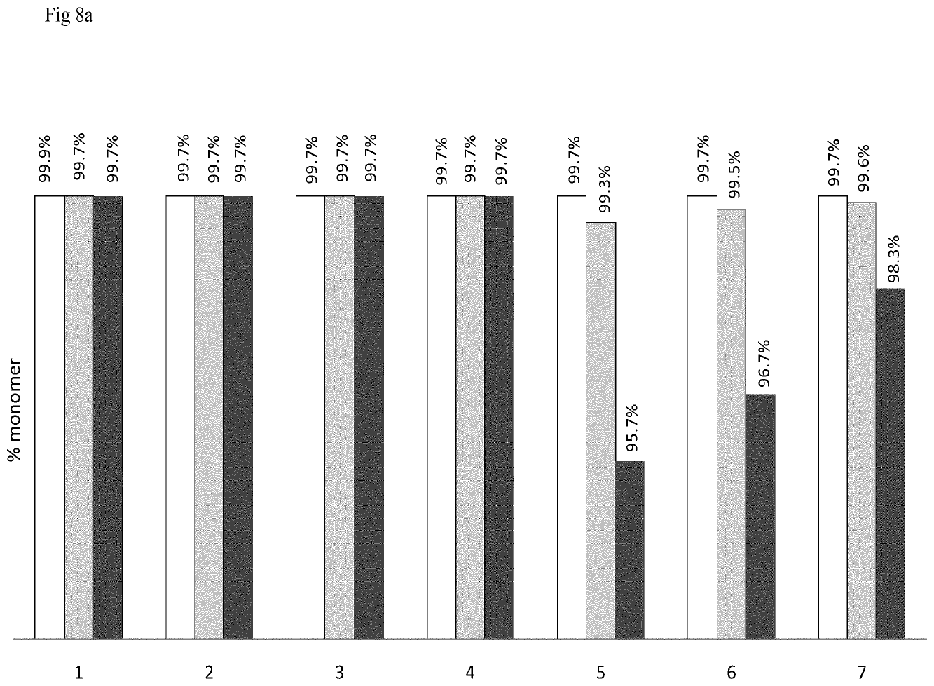

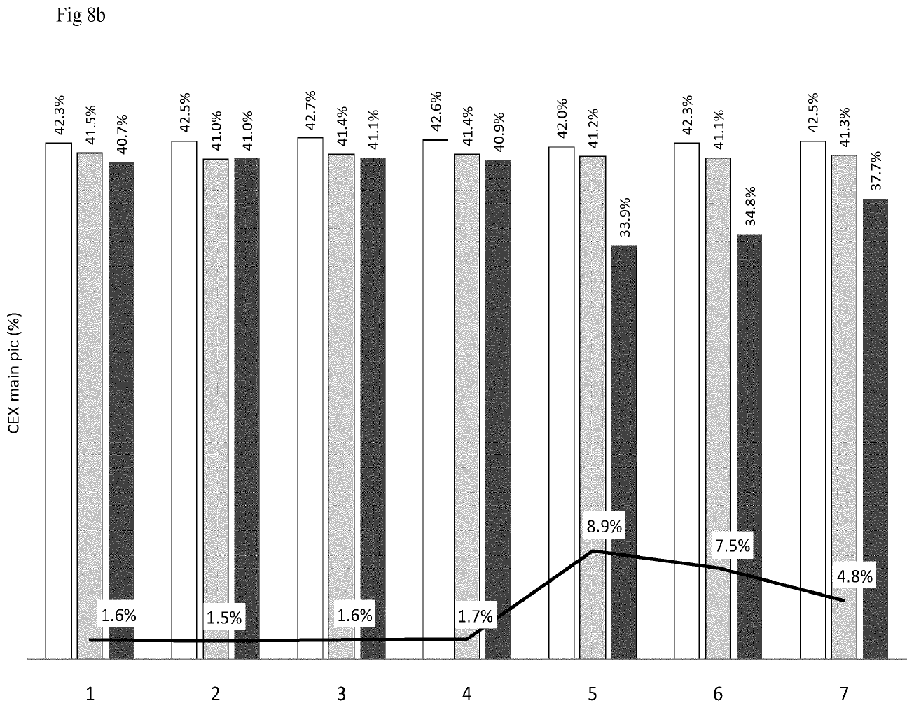

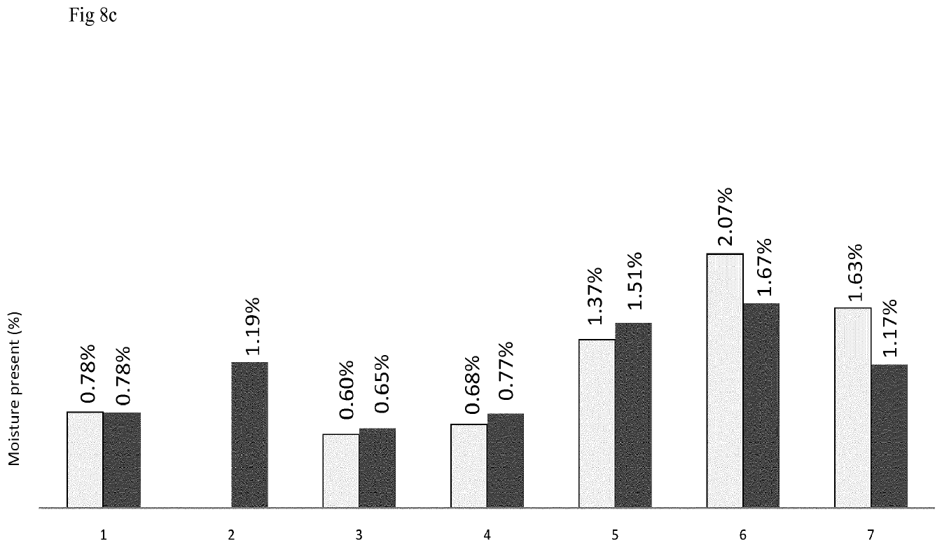

The results of the stability study at 40° C. for one month can be seen in FIGS. 8a, 8b and 8c. Formulations 1-4 showed a high percentage of monomers after one month (FIG. 8a) as well as similar CEX profiles (FIG. 8b). The percentage of moisture measured in the lyophilized cakes was shown to be lowest for formulations 1-4. One the basis of these results, formulations 2 and 4 were selected for use in further screens.

2.7: Screen 7

### Aim:

To optimise the lyophilization cycle and improve cake appearance by reducing the crack and shrinkage observed during secondary drying.

### Materials & Method:

Formulations 2 and 4 were selected from Screen 6 and these were sent to Telstar (Terrassa, Spain) for determination of Collapse temperature (Tg′).

The secondary drying step of the lyophilization cycle was examined to try and improve cake appearance and reduce the percentage of moisture in the cakes. Two new lyophilization protocols were tested in which the secondary drying step was slowed down. With reference to Table 14, the time for the two secondary drying steps was altered in one cycle from 5 and 24 hours to 15 and 9 hours, respectively and in a second cycle from 5 and 24 hours to 20 and 4 hours respectively, whilst a vacuum pressure of 0.1 mbars was maintained throughout the secondary drying steps.

### Results:

The collapse temperatures of the two formulations tested are shown in Table 15 below, along with the composition of the formulations. Both formulations were considered to have very similar collapse temperatures.

TABLE 15
Selected formulations and their Collapse temperature

| | | Mannitol | | Glycine | | Tg′ |
|---|---|---|---|---|---|---|
| No | Buffer | % | Sucrose % | % | Tween80 ™ % | ° C. |
| 1 | 5.76 mM | 2 | 10 | 0 | 0.014 | −31 |
| | sodium | | | | | |
| 2 | acetate | 0 | 12 | 1 | 0.014 | −32 |
| | pH 5.7 | | | | | |

Following lyophilization with a secondary drying step of 15 and 9 hours, the moisture content was determined to be 0.94% for formulation 1 and 1.19% for formulation 2. Following lyophilization with a secondary drying step of 20 and 4 hours, the moisture content was determined to be 1.20% for formulation 1 and 1.70% for formulation 2.

### 2.8: Screen 8

### Aim:

To determine the stability of the two selected anti-HER2 antibody lyophilized formulations at 40° C. for one month. This study is the same as the one month stability study described in section 2.6 for Screen 6 but repeated due to the changes in the lyophilisation cycle described in section 2.7 above.

### Materials & Method:

The composition of formulations 1 and 2 is shown in Table 16 below. Both formulations were subjected to the lyophilisation cycle shown in Table 17 below, where the secondary drying time was chosen to be 20 and 4 hours. The samples were analysed by SEC and CEX after one and two months storage at 40° C.

TABLE 16
Composition of formulations 1 and 2

| No | Buffer | Mannitol % | Sucrose % | Glycine % | Tween80 ™ % |
|---|---|---|---|---|---|
| 1 | 5.76 mM | 2 | 10 | 0 | 0.014 |
| 2 | sodium | 0 | 12 | 1 | 0.014 |
| | acetate | | | | |
| | pH 5.7 | | | | |

TABLE 17
Optimized lyophilisation cycle parameters

| Step | Temp (° C.) | Vacuum (mbar) | Time (h) |
|---|---|---|---|
| Freezing | 5 | | 0.20 |
| Freezing | 5 | | 0.30 |
| Freezing | 0 | | 0.05 |
| Freezing | 0 | | 0.30 |
| Freezing | −45 | | 0.45 |
| Freezing | −45 | | 2.00 |
| Freezing/annealing | −15 | | 0.30 |
| Freezing/annealing | −15 | | 2.00 |
| Freezing | −45 | | 0.30 |
| Freezing | −45 | | 2.00 |
| Vacuum | | 0.1 | |
| Primary drying | −30 | 0.1 | 1.00 |
| Primary drying | −30 | 0.1 | 72.00 |
| Secondary drying | 25 | 0.1 | 20.00 |
| Secondary drying | 25 | 0.1 | 4.00 |

### Results:

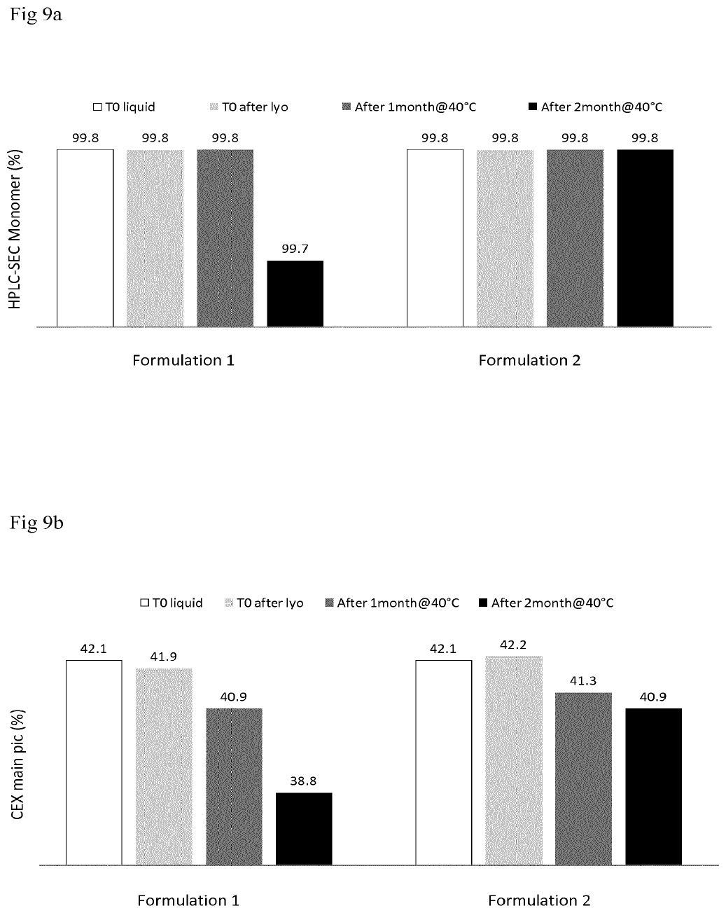

The percentage of monomers in both candidate formulations after one and two months as measured by HPLC-SEC can be seen in FIG. 9a. The HPLC-CEX results are shown in FIG. 9b. At time 0, for the liquid and lyophilized formulations, there is little difference in stability for Formulations 1 and 2. After one month storage at 40° C., there is no difference between the formulations following HPLC-CEX; however there is a small difference shown by HPLC-SEC in FIG. 9a. It can be clearly seen from both FIGS. 9a and 9b that after two months storage, Formulation 2 is more stable than Formulation 1. Formulation 2 was then chosen to be taken forward for use in a long term stability study.

### Example 3—Long Term Stability Study

### Aim:

The purpose of this stability study was to determine the stability of the lyophilized anti-HER2 antibody formulation at a target concentration of 30 g/L, for intended storage at 5±3° C., an accelerated study at 25±2° C. and a stress study 40±2° C. The stability of the lyophilized anti-HER2 antibody formulation will be followed for 36 months at 5±3° C., 12 months at 25±2° C. and 3 months at 40±2° C.

### Materials & Method:

The formulation buffer was 5.75 mM Sodium Acetate pH 5.7 (Merck), 12% Sucrose (Sigma), 1% glycine (Merck) and 0.014% Tween80™ (VWR). Hydrochloric Acid 37% (Sigma) was used for pH adjustment. Specifications for the buffer were 5.7±0.1 for the pH and 0.42 to 0.66 mS/cm for the conductivity. All chemicals were suitable for parenteral administration. The buffer was then filtered through a 0.22 μm filter into a bag.

Stock solutions of the three families of excipients, lyoprotectant (sucrose), bulking agent (glycine) and surfactant (Tween80™) were prepared in 5.75 mM sodium acetate buffer pH 5.7. Stock solutions of sucrose, glycine and Tween80™ were prepared at concentrations of 600 g/L, 130 g/L and 100 g/L, respectively. The excipients were then added to the anti-HER2 antibody formulation by dilution of the stock solution in the formulation to achieve the desired concentration.

Purified anti-HER2 antibody was diafiltrated 7-10 times by tangential flow filtration (TFF) in 5.75 mM sodium acetate pH 5.7 and then concentrated to 50-55 g/L. After TFF, the anti-HER2 antibody was formulated by the addition of sucrose, glycine and Tween80™ to reach the final buffer composition. The reference standard used was DRS-anti-HER2 antibody-01 used for the analytical testing and kept under Quality Control at a temperature of below −60° C.

To prepare the lyophilized drug product, a Telstar lyophilization system was used (Lyobeta 15 lyophilizer; Telstar, Terrassa, Spain). A VP600 Vötsch stability chamber (Vötsch Industrietechnik GmbH, Germany) is used for the 3 months study at 40±2° C., ±5% relative humidity test and also for the 12 months study at 25±2° C., ±5% relative humidity test. A standard laboratory fridge is used for the 36 months stability at 5±3° C.

Vials and stopper materials used were those intended for clinical trial manufacture. Vials were 20 ml Fiolax clear, USP type I glass vials purchased from Schott (Art. No: 1156521) and the stoppers were Flurotec distributed by West Pharma (ref FD20TT3WRS). All vials were crimped.

The formulated anti-HER2 antibody was aliquotted in 20 ml Schott vials at a volume of 5 ml per vial. A total of 62 vials were prepared. The rest of the shelf was filled with placebo vials. The vials were lyophilized according to the parameters in Table 18 below.

TABLE 18
Lyophilisation cycle parameters for the long term stability study

| Step | Temp (° C.) | Vacuum (mbar) | Time (h) |
|---|---|---|---|
| Freezing | 5 | | 0.20 |
| Freezing | 5 | | 0.30 |
| Freezing | 0 | | 0.05 |
| Freezing | 0 | | 0.30 |
| Freezing | −45 | | 0.45 |
| Freezing | −45 | | 2.00 |
| Freezing/annealing | −15 | | 0.30 |
| Freezing/annealing | −15 | | 2.00 |
| Freezing | −45 | | 0.30 |
| Freezing | −45 | | 2.00 |
| Vacuum | | 0.1 | |
| Primary drying | −30 | 0.1 | 1.00 |
| Primary drying | −30 | 0.1 | 72.00 |
| Secondary drying | 25 | 0.1 | 20.00 |
| Secondary drying | 25 | 0.1 | 4.00 |

Stability of the drug product was determined using a number of tests to evaluate changes in the drug product over time. CEX-HPLC (Dionax, ProPac WCX-104×250 mm), SEC-HPLC (Phenomenex, YARRA 3u SEC-3000 7.8×300 mm) and SDS-PAGE are sensitive indicators of product breakdown. The profile of drug product using these assays will change over time if the sample is subject to degradation. The A280 assay can be used to detect changes in the protein level which may be indicative of product breakdown or adhesion to the container wall. An ELISA cell based assay and an ELISA binding assay can be used to determine the potency of the product. A change in the potency of the sample with time is indicative of product deterioration. The pH of the formulation will also be checked over time to determine pH stability and the physical appearance of the solution also monitored. The presence or absence of sub visible particles after dissolution of the cake will also be checked. Any variation in moisture of the product will be monitored. This can increase the sensitivity of the lyophilized product to degradation and will be checked by a Karl Fisher titration. Finally, reconstitution time will be measured and used to determine the complete dissolution time of the lyophilized product cakes. These parameters will be assessed according to the criteria shown in Table 19 below for the time points for the temperatures 5° C., 25° C. and 40° C.

TABLE 19
Parameters for assessing stability of
the anti-HER2 antibody drug product

| Parameter | Specifications |
|---|---|
| SEC - HPLC | monomer >98% |
| CEX-HPLC | comparable to reference standard* |
| SDS-PAGE | comparable to reference standard* |
| A280 | ≥20 and ≤24 mg/ml |
| ELISA Binding | ≥75% and ≤125% of EC₅₀ of reference standard* |
| pH | ≥5.6 and ≤5.8 |
| Appearance | Colorless, clear to slightly opalescent liquid |
| | Practically free of visible particulates |
| Reconstitution Time | ≤3 min |
| Sub visible particles | ≤6000 particles ≥10 μm/vials |
| | ≤600 particles ≥25 μm/vials |
| Karl Fisher titration | ≤2% |

*Reference standard used was DRS-anti-HER2 antibody-01

### Results:

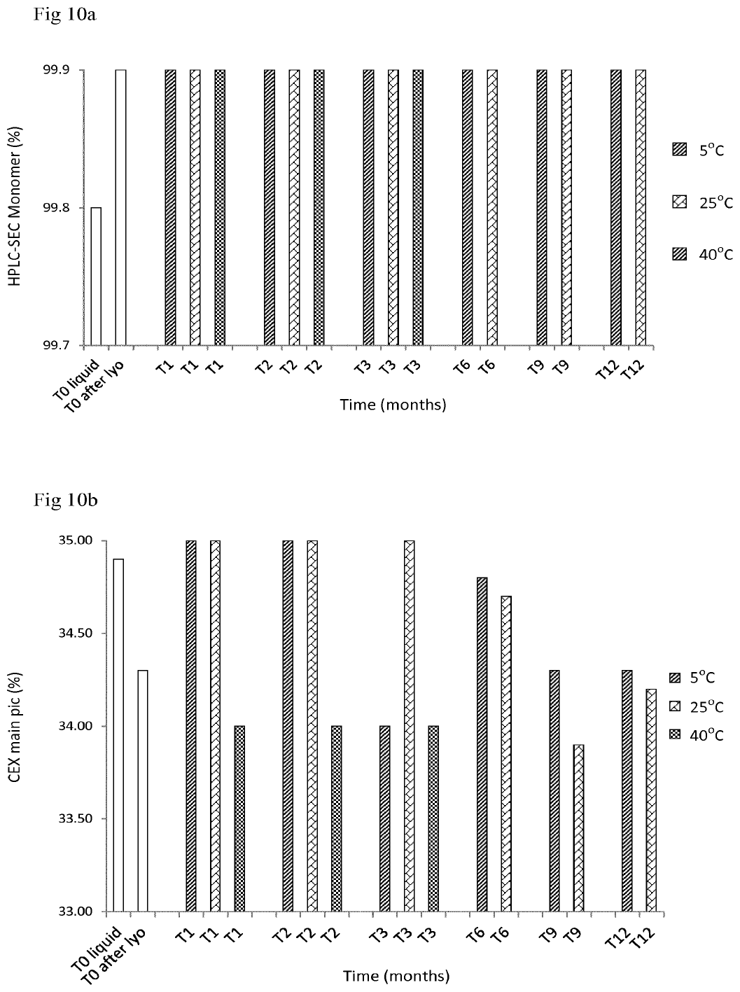

The percentage of monomers in the lyophilized anti-HER2 antibody formulation after 12 months as measured by HPLC-SEC is shown in FIG. 10a. This figure shows that there is no difference in percentage monomer in the formulation samples at all time points and at all temperatures tested. The HPLC-CEX results for 12 months are shown in FIG. 10b. The large scale used on the y-axis of the graph gives the impression of a large difference between the stability of the formulation samples, particularly at 40° C. and later time points; however there is actually only a difference at the most of approximately 1.5% and the formulation was considered stable for the duration of the 12 month study. The results for the other parameters tested are summarised in Table 20 below where it is indicated whether the specification for each assessment parameter was met.

For most of the parameters tested, the anti-HER2 antibody formulation tested was found to be within the specifications as given in Table 19, for all temperatures and time points tested up to and including 12 months. For the A280 assay, used to detect changes in the protein level which may be indicative of product breakdown or adhesion to the container wall, the initial assessment specification of ≥20 and ≤24 mg/ml was miscalculated. At the start of the stability study the result for the A280 assay was actually 18 mg/ml (already outside of the assessment specification) and this figure remained the same throughout the study, indicating that there was actually no product breakdown or adhesion to the container wall. For the assessment of pH, the specification of a pH of ≥5.6 and ≤5.8 were set. For the readings at the 6 month time point at 5° C. and 25° C., the pH values recorded were 5.865, only fractionally outside of the pH range set. At all other time points and temperatures tested the pH specifications were within the range set.

In conclusion, this stability study over 12 month has demonstrated that the anti-HER2 antibody formulation of the present invention has met the majority of the assessment parameters for stability and is therefore expected to be stable for the remaining duration of the long term stability study.

TABLE 20
Summary of stability assessment parameters for the anti-HER2 antibody drug product
at the times and temperatures tested

| | | | Month | | | | | | | | | | | | | | |
|---|---|---|---|---|---|---|---|---|---|---|---|---|---|---|---|---|---|
| | | | 1 | | | 2 | | | 3 | | | 6 | | 9 | | 12 | |
| | 0 | 0 | Temp ° C. | | | | | | | | | | | | | | |
| | liquid | after ly0 | 5 | 25 | 40 | 5 | 25 | 40 | 5 | 25 | 40 | 5 | 25 | 5 | 25 | 5 | 25 |
| Parameter | | | | | | | | | | | | | | | | | |
| SEC-HPLC | ✓ | ✓ | ✓ | ✓ | ✓ | ✓ | ✓ | ✓ | ✓ | ✓ | ✓ | ✓ | ✓ | ✓ | ✓ | ✓ | ✓ |
| CEX-HPLC | ✓ | ✓ | ✓ | ✓ | ✓ | ✓ | ✓ | ✓ | ✓ | ✓ | ✓ | ✓ | ✓ | ✓ | ✓ | ✓ | ✓ |
| SDS-PAGE | ✓ | ✓ | ✓ | ✓ | ✓ | ✓ | ✓ | ✓ | ✓ | ✓ | ✓ | ✓ | ✓ | ✓ | ✓ | ✓ | ✓ |
| A280 | x | x | x | x | x | x | x | x | x | x | x | x | x | x | x | x | x |
| ELISA | x | ✓ | ✓ | ✓ | x | ✓ | ✓ | ✓ | ✓ | ✓ | ✓ | ✓ | ✓ | ✓ | ✓ | ✓ | ✓ |
| Binding | | | | | | | | | | | | | | | | | |
| pH | ✓ | ✓ | ✓ | ✓ | ✓ | ✓ | ✓ | ✓ | ✓ | ✓ | ✓ | x | x | ✓ | ✓ | ✓ | ✓ |
| Appearance | ✓ | ✓ | ✓ | ✓ | ✓ | ✓ | ✓ | ✓ | ✓ | ✓ | ✓ | ✓ | ✓ | ✓ | ✓ | ✓ | ✓ |
| Reconstitution | N/A | N/A | ✓ | ✓ | ✓ | ✓ | ✓ | ✓ | ✓ | ✓ | ✓ | ✓ | ✓ | ✓ | ✓ | ✓ | ✓ |
| Time | | | | | | | | | | | | | | | | | |
| Sub Visible | nd | ✓ | nd | nd | nd | nd | nd | nd | nd | nd | nd | nd | nd | nd | nd | ✓ | ✓ |
| particles | | | | | | | | | | | | | | | | | |
| Karl Fisher | nd | ✓ | nd | nd | nd | nd | nd | nd | nd | nd | ✓ | nd | nd | nd | nd | ✓ | ✓ |
| titration | | | | | | | | | | | | | | | | | |

✓: parameter met.
x: parameter unmet.
nd: not determined.
N/A: not applicable

1. A pharmaceutical formulation comprising an anti-HER2 antibody or a fragment thereof that recognizes and binds to human HER2, one or more pharmaceutically acceptable excipients and an acetate buffer.

2. The pharmaceutical formulation according to claim 1, wherein the pharmaceutical formulation is a liquid formulation, a lyophilized formulation or a reconstituted formulation.

3. The pharmaceutical formulation according to claim 1, wherein said anti-HER2 antibody or a fragment thereof that recognizes and binds to human HER2, is present within said pharmaceutical formulation in an amount of between 10 and 40 mg/ml.

4. (canceled)

5. (canceled)

6. The pharmaceutical formulation according to claim 1, wherein the acetate buffer is sodium acetate buffer present within the pharmaceutical formulation in an amount of between 1 and 10 mM.

7. The pharmaceutical formulation according to claim 1 wherein the pharmaceutically acceptable excipient comprises one or more of a surfactant, lyoprotectant, bulking agent, inorganic or organic salt, stabilizer, diluent, solubilizer, reducing agent, antioxidant, chelating agent and/or preservative.

8. (canceled)

9. The pharmaceutical formulation according to claim 7, wherein the surfactant is Polysorbate 80 present within the pharmaceutical formulation in an amount of between 0.001% and 0.1% (w/w).

10. (canceled)

11. The pharmaceutical formulation according to claim 1, wherein the lyoprotectant is sucrose present within the pharmaceutical formulation in an amount of between 5% and 15% (w/w).

12. The pharmaceutical formulation according to claim 7, wherein the lyoprotectant is sucrose and mannitol present within the pharmaceutical formulation in an amount of between 5% and 15% (w/w) and 0.5% and 5% (w/w), respectively.

13. The pharmaceutical formulation according to claim 7, wherein the lyoprotectant is sucrose present within the pharmaceutical formulation in a molar ratio of lyoprotectant to antibody of between 1500 to 2000 moles sucrose to 1 mole antibody.

14. (canceled)

15. The pharmaceutical formulation according to claim 7, wherein the bulking agent is glycine present within the pharmaceutical formulation in an amount of between 0.5% and 1.5% (w/w).

16. (canceled)

17. (canceled)

18. (canceled)

19. A lyophilized pharmaceutical formulation according to claim 1 which comprises 30 mg/ml of an anti-HER2 antibody or a fragment thereof that recognizes and binds to human HER2, 12% (w/w) sucrose, 1% (w/w) glycine and 0.014% (w/w) Polysorbate 80 and 5.75 mM sodium acetate buffer, wherein the pH of the formulation is 5.7±0.2.

20. (canceled)

21. (canceled)

22. The pharmaceutical formulation according to claim 19, wherein the formulation comprises a diluent and is a reconstituted formulation.

23. (canceled)

24. The pharmaceutical formulation according to claim 22, which comprises 21 mg/ml of an anti-HER2 antibody or a fragment thereof that recognizes and binds to human HER2, 8.4% (w/w) sucrose, 0.7% (w/w) glycine, 0.01% (w/w) Polysorbate 80, WFI and 4 mM sodium acetate, wherein the pH of the formulation is 5.7±0.2.

25. (canceled)

26. The pharmaceutical formulation according to claim 1, wherein the anti-HER2 antibody or a fragment thereof that recognizes and binds to human HER2, has a heavy chain of SEQ ID NO: 10 and a light chain of SEQ ID NO: 11.

27. (canceled)

28. (canceled)

29. A method of treatment of a HER2 related disorder which comprises administering to a subject a therapeutically effective amount of a pharmaceutical formulation according to claim 1, wherein the HER2 related disorder is selected from a HER2 positive cancer such as metastatic breast cancer, early breast cancer and metastatic gastric cancer.

30. (canceled)

31. (canceled)

32. An article of manufacture comprising:
(a) a container which holds a lyophilized formulation according to claim 19; and
(b) instructions for reconstituting the lyophilized formulation with a diluent to an antibody concentration in the reconstituted formulation of about 10 to 40 mg/ml.
